SEQUENCE LISTING (42 sequences)

SEQ ID NO: 1 - protein, 28 aa, Actinia fragacea
SADVAGAVIDGAGLGFDVLKTVLEALGN

SEQ ID NO: 2 - protein, 44 aa, Escherichia coli
QDLDEVDAGSMTEIVADKTVEVVKNAIETADGALDLYNKYLDQV

SEQ ID NO: 3 - protein, 18 aa, Yersinia enterocolitica
FTGAIGGIIAMAITGGIF

SEQ ID NO: 4 - protein, 42 aa, Yersinia enterocolitica
LVDAFKDLIPTGENLSELDLAKPEIELLKQSLEITKKLLGQF

SEQ ID NO: 5 - protein, 35 aa, Escherichia coli
APLVRWNRVISQLVPTISGVHDMTETVRYIKRWPN

SEQ ID NO: 6 - protein, 22 aa, synthetic construct
  REGION 1..22: note = TM sequence
VHGNAEVHASFFDIGGSVSAGF

SEQ ID NO: 7 - protein, 28 aa, synthetic construct
  REGION 1..28: note = TM sequence with linkers
GSSVHGNAEVHASFFDIGGSVSAGFSSG

SEQ ID NO: 8 - protein, 37 aa, Thermoplasma acidophilum
PVPDPVKEKEKEERKKQQEKEEKEEKKKGDEDDKGPP

SEQ ID NO: 9 - protein, 251 aa, synthetic construct
  REGION 1..251: note = subunit of artificial PA28 nanopore
MATLRVHPEAQAKVDVFREDLCSKTENLLGSYFPKKISELDAFLKEPALNEANLSNLKAPLDIGSSVHGNAEVHASFFDIGGSVSAGFSSGCGPVNCNEKIVVLLQRLKPEIKDVTEQLNLVTTWLQLQIPRIEDGNNFGVAVQEKVFELMTNLHTKLEGFHTQISKYFSERGDAVAKAAKQPHVGDYRQLVHELDEAEYQEIRLMVMEIRNAYAVLYDIILKNFEKLKKPRGETKGMIYGSSWSHPQFEK

SEQ ID NO: 10 - protein, 260 aa, Thermoplasma acidophilum
MATLRVHPEAQAKVDVFREDLCSKTENLLGSYFPKKISELDAFLKEPALNEANLSNLKAPLDIPVPDPVKEKEKEERKKQQEKEEKEEKKKGDEDDKGPPCGPVNCNEKIVVLLQRLKPEIKDVTEQLNLVTTWLQLQIPRIEDGNNFGVAVQEKVFELMTNLHTKLEGFHTQISKYFSERGDAVAKAAKQPHVGDYRQLVHELDEAEYQEIRLMVMEIRNAYAVLYDIILKNFEKLKKPRGETKGMIYGSSWSHPQFEK

SEQ ID NO: 11 - protein, 11 aa, Escherichia coli
AANDENYALAA

SEQ ID NO: 12 - protein, 123 aa, synthetic construct
  REGION 1..123: note = Substrate S1
MGHHHHHHSSRRRRRRRRRRSSSSSSSSSSSSSSSFGYGWSSSSSSSSSSSSSSSRRRRRRRRRRSSSSSSSSSSSSSSSFGYGWSSSSSSSSSSSSSSSRRRRRRRRRRSSAANDENYALAA

SEQ ID NO: 13 - protein, 210 aa, synthetic construct
  REGION 1..210: note = Substrate S2
MGHHHHHHSSRRRRRPVPLPIPVPLPIPVPLPIPRRRRRSSSSSSSSSSSSSSSSSSSSSSSSSSSSSSSSSSSSSSSSSSSSSSSSSSRRRRRPVPLPIPVPLPIPVPLPIPRRRRRSSSSSSSSSSSSSSSSSSSSSSSSSSSSSSSSSSSSSSSSSSSSSSSSSSEEEEEPVPLPIPVPLPIPVPLPIPEEEEESSAANDENYALAA

SEQ ID NO: 14 - protein, 260 aa, synthetic construct
  REGION 1..260: note = Substrate S3
MGHHHHHHSSSKGEELFTGVVPILVELDGDVNGHKFSVSGEGEGDATYGKLTLKFICTTGKLPVPWPTLVTTLTYGVQCFSRYPDHMKRHDFFKSAMPEGYVQERTISFKDDGNYKTRAEVKFEGDTLVNRIELKGIDFKEDGNILGHKLEYNYNSHNVYITADKQKNGIKANFKIRHNIEDGSVQLADHYQQNTPIGDGPVLLPDNHYLSTQSALSKDPNEKRDHMVLLEFVTAAGITHGMDELYKSSAANDENYALAA

SEQ ID NO: 15 - protein, 5 aa, synthetic construct
  REGION 1..5: note = Linker 1
GSSSE

SEQ ID NO: 16 - protein, 5 aa, synthetic construct
  REGION 1..5: note = Linker 2
SNSSG

SEQ ID NO: 17 - protein, 32 aa, synthetic construct
  REGION 1..32: note = TM sequence with linkers
GSSSEVHGNAEVHASFFDIGGSVSAGFSNSSG

SEQ ID NO: 18 - protein, 12 aa, Thermoplasma acidophilum
SVDAESGKTKGG

SEQ ID NO: 19 - protein, 263 aa, synthetic construct
  REGION 1..263: note = N-terminally Strep-tagged subunit artificial PA26-nanopore
MGWSHPQFEKSSGPPKRAALIQNLRDSYTETSSFAVIEEWAAGTLQEIEGIAKAAAEAHGVIRNSTYGRAQAEKSPEQLLGVLQRYQDLCHNVYCQAETIRTVIAIRIPEHKEEDNLGVAVQHAVLKIIDELEIKTLGSGEKSGSGGAPTPIGMYALREYLSARSTVEDKLLGGSSSEVHGNAEVHASFFDIGGSVSAGFSNSSGSQSPSLLLELRQIDADFMLKVELATTHLSTMVRAVINAYLLNWKKLIQPRTGSDHMVS

SEQ ID NO: 20 - protein, 243 aa, synthetic construct
  REGION 1..243: note = N-term S-tagged PA26 nanopore with del seq
MGWSHPQFEKSSGPPKRAALIQNLRDSYTETSSFAVIEEWAAGTLQEIEGIAKAAAEAHGVIRNSTYGRAQAEKSPEQLLGVLQRYQDLCHNVYCQAETIRTVIAIRIPEHKEEDNLGVAVQHAVLKIIDELEIKTLGSGEKSGSGGAPTPIGMYALREYLSARSTVEDKLLGSVDAESGKTKGGSQSPSLLLELRQIDADFMLKVELATTHLSTMVRAVINAYLLNWKKLIQPRTGSDHMVS

SEQ ID NO: 21 - protein, 5 aa, synthetic construct
  REGION 1..5: note = linker
SSSSS

SEQ ID NO: 22 - protein, 32 aa, synthetic construct
  REGION 1..32: note = TM sequence with linkers
SSSSSVHGNAEVHASFFDIGGSVSAGFSSSSS

SEQ ID NO: 23 - protein, 9 aa, Aquifex sp.
  source 1..9: note = Aquifex aeolicus
KGAFTGAVS

SEQ ID NO: 24 - protein, 302 aa, synthetic construct
  REGION 1..302: note = N-term S-tag artificial ATPase with TM sequence
MGWSHPQFEKSSGRKENELLRREKDLKEEEYVFESPKMKEILEKIKKISCAECPVLITGESGVGKEVVARLIHKLSDRSKEPFVALNVASIPRDIFEAELFGYESSSSSVHGNAEVHASFFDIGGSVSAGFSSSSSSKEGFFELADGGTLFLDAIGELSLEAQAKLLRVIESGKFYRLGGRKEIEVNVRILAATNRNIKELVKEGKFREDLYYRLGVIEIEIPPLRERKEDIIPLANHFLKKFSRKYAKEVEGFTKSAQELLLSYPWYGNVRELKNVIERAVLFSEGKFIDRGELSCLVNSK

SEQ ID NO: 25 - protein, 279 aa, synthetic construct
  REGION 1..279: note = N-term S-tag artificial ATPase nanopore
MGWSHPQFEKSSGRKENELLRREKDLKEEEYVFESPKMKEILEKIKKISCAECPVLITGESGVGKEVVARLIHKLSDRSKEPFVALNVASIPRDIFEAELFGYEKGAFTGAVSSKEGFFELADGGTLFLDAIGELSLEAQAKLLRVIESGKFYRLGGRKEIEVNVRILAATNRNIKELVKEGKFREDLYYRLGVIEIEIPPLRERKEDIIPLANHFLKKFSRKYAKEVEGFTKSAQELLLSYPWYGNVRELKNVIERAVLFSEGKFIDRGELSCLVNSK

SEQ ID NO: 26 - protein, 462 aa, synthetic construct
  REGION 1..462: note = C-terminally Strep tagged subunit of artificial ClpP-nanopore
MGSYSGERDNFAPHMALVPMVIEQTSRGERSFDIYSRLLKERVIFLTGQVEDHMANLIVAQMLFLEAENPEKDIYLYINSPGGVITAGMSIYDTMQFIKPDVSTICMGQAASMGAFLLTAGAKGKRFCLPNSRVMIHQPLGGYQGQATDIEIHAREILKVKGRMNELMALHTGQSLEQIERDTERDRFLSAPEAVEYGLVDSILTHRNATLRVHPEAQAKVDVFREDLCSKTENLLGSYFPKKISELDAFLKEPALNEANLSNLKAPLDIGSSSEVHGNAEVHASFFDIGGSVSAGFSNSSGCGPVNCNEKIVVLLQRLKPEIKDVTEQLNLVTTWLQLQIPRIEDGNNFGVAVQEKVFELMTNLHTKLEGFHTQISKYFSERGDAVAKAAKQPHVGDYRQLVHELDEAEYQEIRLMVMEIRNAYAVLYDIILKNFEKLKKPRGETKGMIYGSSWSHPQFEK

SEQ ID NO: 27 - protein, 30 aa, synthetic construct
  REGION 1..30: note = TM region beta strand
FDIGGSVSAGFSNSNSSTVAIDHSLSLSSG

SEQ ID NO: 28 - protein, 30 aa, synthetic construct
  REGION 1..30: note = TM region beta strand
GSSRTISKNTSTSRTHTSEVHGNAEVHASF

SEQ ID NO: 29 - protein, 7 aa, synthetic construct
  REGION 1..7: note = Linker sequence d4 mutant
GSSHTSE

SEQ ID NO: 30 - protein, 7 aa, synthetic construct
  REGION 1..7: note = Linker sequence d4 mutant
SNSNSSG

SEQ ID NO: 31 - protein, 5 aa, synthetic construct
  REGION 1..5: note = linker sequence d2 mutant
GSSSE

SEQ ID NO: 32 - protein, 5 aa, synthetic construct
  REGION 1..5: note = linker sequence d2 mutant
SNSSG

SEQ ID NO: 33 - protein, 10 aa, Homo sapiens
DRVYIHPFHL

SEQ ID NO: 34 - protein, 17 aa, Homo sapiens
YGGFLRRIRPKLKWDNQ

SEQ ID NO: 35 - protein, 7 aa, synthetic construct
  REGION 1..7: note = part of S2
PVPLPIP

SEQ ID NO: 36 - protein, 19 aa, synthetic construct
  REGION 1..19: note = part of substrate 2
PVPLPIPVPLPIPVPLPIP

SEQ ID NO: 37 - protein, 8 aa, synthetic construct
WSHPQFEK

SEQ ID NO: 38 - protein, 60 aa, synthetic construct
GSSRTISKNTSTSRTHTSEVHGNAEVHASFFDIGGSVSAGFSNSNSSTVAIDHSLSLSSG

SEQ ID NO: 39 - protein, 102 aa, synthetic construct
HHHHHHRRRRRRRRRRSSSSSSSSSSSSSSSFYWSSSSSSSSSSSSSSSRRRRRRRRRRSSSSSSSSSSSSSSSFYWSSSSSSSSSSSSSSSRRRRRRRRRR

SEQ ID NO: 40 - protein, 193 aa, synthetic construct
HHHHHHRRRRRPVPLPIPVPLPIPVPLPIPRRRRRSSSSSSSSSSSSSSSSSSSSSSSSSSSSSSSSSSSSSSSSSSSSSSSSSSRRRRRPVPLPIPVPLPIPVPLPIPRRRRRSSSSSSSSSSSSSSSSSSSSSSSSSSSSSSSSSSSSSSSSSSSSSSSSSSEEEEEPVPLPIPVPLPIPVPLPIPEEEEE

SEQ ID NO: 41 - protein, 15 aa, synthetic construct
SSSSSSSSSSSSSSS

SEQ ID NO: 42 - protein, 10 aa, synthetic construct
RRRRRRRRRR

(19) United States
(12) Patent Application Publication
(10) Pub. No.: US 2024/0288416 A1
(43) Pub. Date: Aug. 29, 2024

(54) ARTIFICIAL NANOPORES AND USES AND METHODS RELATING THERETO

(71) Applicant: Rijksuniversiteit Groningen, Groningen (NL)

(72) Inventors: Giovanni MAGLIA, Groningen (NL); Shengli ZHANG, Shanghai (CN)

(21) Appl. No.: 18/394,833

(22) Filed: Dec. 22, 2023

Related U.S. Application Data
(63) Continuation of application No. 17/777,757, filed on May 18, 2022.

(30) Foreign Application Priority Data
Nov. 19, 2019 (EP) 19210168.1

(51) Int. Cl. G01N 33/487; B82Y 15/00; C12Q 1/6869

(52) U.S. Cl. CPC G01N 33/48721; B82Y 15/00; C12Q 1/6869

(57) ABSTRACT

The invention relates to the field of nanopores and the use thereof in analyzing biopolymers, including polypeptides and polynucleotides. Provided is an artificial nanopore comprising a multimeric assembly of subunits, each subunit comprising (i) the transmembrane (TM) sequence of a β-barrel or α-helical pore forming protein fused to the amino acid sequence of (ii) a subunit of a ring-forming protein capable of controlling the transport of a polypeptide or polynucleotide across the TM region of the assembly.

### CROSS-REFERENCE

This application is a continuation of U.S. application Ser. No. 17/777,757, filed May 18, 2022, which is a 371 national phase entry of International Application No. PCT/NL2020/050726, filed Nov. 19, 2019, each of which is entirely incorporated herein by reference.

### SEQUENCE LISTING

The instant application contains a Sequence Listing which has been submitted electronically in XML format and is hereby incorporated by reference in its entirety. Said XML copy, created on Dec. 22, 2023, is named 64828-703_301_SL.xml and is 49,725 bytes in size.

### BACKGROUND

The invention relates generally to the field of nanopores and the use thereof in analyzing biopolymers and other (biological) compounds. In particular, it relates to artificial nanopores and multi-protein assemblies thereof, and their application in single molecule analysis, such as single molecule polypeptide sequencing.

### DETAILED DESCRIPTION

In cells, splicing and post translational modifications induce a large heterogeneity in protein populations that is not easily addressed by ensemble techniques. However, today no technique exists that allows the sequencing of single proteins. Biological nanopores are emerging as powerful single-molecule tools.

The ionic current passing through proteins that form nanoscale apertures on biological membranes are emerging as powerful single-molecule tools. Compared to nanopores formed on solid-state membranes, biological nanopores have the advantage that they self-assemble with atomic precision and they can interface with nature's nanomachines, which evolved over billions years to handle biomolecules.

Most notably, nanopores aided by DNA-processing enzymes are now used to sequence DNA^{1,2}. Recently we have shown²⁹ that octameric Fragaceatoxin C (FraC) nanopores from the sea anemone Actinia fragacea can be used to study proteins and peptides, and that at low pH (i.e. pH 3.8) the ionic signal from peptide blockades to a FraC nanopore relate directly to the volume of the peptide. See also WO2018/012963 in the name of the applicant.

The identification and sequencing of proteins will require designing and engineering new nanopores that are capable of controlling the transit of polypeptides. However, because the folding and assembly of proteins cannot be easily predicted, building at the nanoscale using polypeptides remains extremely challenging. To date, even the design of a protein nanopore that can remain open in the lipid bilayer has yet to be reported, let alone the preparation of nanopores with advanced functions. The ability to design artificial nanopores coupled to complex molecular machines made entirely of proteins would then expand the use of biological nanopores in nanotechnology, and would elucidate fundamental questions about membrane protein structure. The fabrication of complex protein structures would address emerging challenges in nanoscale assembly. The building of a robust transmembrane machine is, therefore, an important goal in nanotechnology.

In the mainstream approach to single-molecule protein sequencing, proteins are unfolded and processively translocated across a nanopore. In an important proof of concept work (Nivala et al., Nat Biotechnol. 2013 March; 31(3):47-250) proteins elongated by a N-terminal polypeptide were partially threaded across an α-HL nanopore, while a ClpX unfoldase present as soluble protein on the other side of the pore forcefully translocated the proteins by unfolding them against the entry of the nanopore. Although proteins domains could be recognized, the complex current signature arising from the unfolding process prevented the recognition of polypeptides sequences. In another approach²⁹, proteins might be cleaved at specific sites and nanopore currents used to identify the released peptides.

Therefore, the present inventors aimed at designing and engineering new, protein-based nanopores that are capable (as part of a multi-protein sensor complex) of unfolding proteins, controlling their processive and unidirectional transit across the nanopore, and recognize proteins by ionic currents.

It was surprisingly shown that upon the introduction of a protease directly above a nanopore, peptides are captured and read as soon as they are released, thereby providing an artificial nanopore that is advantageously used to sequence protein in solution. More in particular, the inventors designed and produced a stable and low-noise β-barrel nanopore, that is hermetically connected to the 20S proteasome from Thermoplasma acidophilum. The latter is a multi-subunit protease that degrades polypeptides at a variety of conditions including high salt, high temperature and low pH. Surprisingly, a multi-protein assembly comprising the artificial nanopore allowed the docking of unfoldases, which linearized and fed selected proteins into the proteasome chamber without influencing the nanopore signal. In the cut-and-read mode, unfolded polypeptides were first degraded by the proteasome and then recognized by ionic currents. In the thread-and-read mode, an unfoldase threaded intact substrates across the inactivated proteasome and through the nanopore. The linearized substrate are then recognized by the specific modulation of the nanopore current. This integrated molecular sensor has numerous applications e.g. in DNA or protein sequencing and identification.

Accordingly, the invention provides an artificial nanopore comprising an assembly of proteinaceous subunits, each subunit comprising:
(i) the transmembrane (TM) amino acid sequence of a β-barrel or α-helical pore forming protein fused to an amino acid sequence of (ii) a subunit of a ring-forming protein capable of controlling the transport of a polypeptide or polynucleotide across the TM region of the assembly.

Such a nanopore is distinct from the enzyme-pore constructs according to WO2010/004265, disclosing a nanopore made up of alpha-hemolysin covalently attached to a nucleic acid handling enzyme. Specifically disclosed nucleic acid handling enzymes are exonucleases. However, rather than adding a transmembrane region into a circular protein according the present invention, WO2010/004265 describes the fusion of an entire nanopore with a circular protein.

### TM Region

An artificial nanopore as provided herein comprises the TM region of a pore-forming protein. This TM region is formed upon assembly of multiple TM sequences present in each of the subunits, which together form the functional artificial nanopore. Typically, the TM sequence reflects the alternation of hydrophobic and hydrophilic and glycine residues as observed in native transmembrane regions in membrane proteins and pore forming toxins. Pore-forming proteins (PFPs) are usually produced by bacteria, and include a number of protein exotoxins (PFTs, also known as pore-forming toxins) but may also be produced by other organisms such as lysenin, produced by earthworms. They are frequently cytotoxic (i.e., they kill cells), as they create unregulated pores in the membrane of targeted cells. Depending on the secondary structure of the membrane component, PFPs can be classified as α-PFPs, using a ring of amphipathic helices to construct the pore or as β-PFPs, where a β-barrel is used to traverse the membrane.

In one embodiment, the artificial nanopore comprises the TM region of an α-helical pore forming protein. Alpha-pore-forming toxins are well known in the art, and include Haemolysin E family, actinoporins, Corynebacterial porin B, Cytolysin A (ClyA) of E. coli. Preferably, the TM region of FraC, ClyA, AhlB or Wza (translocon for E. coli capsular polysaccharides) is used.

In one aspect, the TM sequence of an actinoporin or actinoporin-like protein is used. Actinoporins (APs) are pore forming toxins from sea anemones (see review by Rojko et al. (BBA, Vol.1858, Issue 3, 2016, Pages 446-456). APs are composed of β-sandwich flanked on two sides by α-helices. The pore is formed by clusters of α-helices. APs are found in about 40 different sea anemone species. To date, the best characterised APs are equinatoxin II (EqtII) from the sea anemone Actinia equina, sticholysin I and II (StnI and StnII) from Stichodactyla helianthus and fragaceatoxin C (FraC) from Actinia fragacea. In one aspect, the TM sequence of FraC is used, which consists of the sequence SADVAGAVIDGAGLGFDVLKTVL EALGN (SEQ ID NO: 1).

In another preferred embodiment, the alpha-helical TM sequence of a member of the ClyA (cytolysin A) protein family is used (PDBs: 2WCD (clya) and 6GY6 (XaxAB).

For example, the TM sequence is QDLDEVDAGSMTEIVADKTVEV VK NAIETADGALDLYNKYLDQV (SEQ ID NO: 2) (ClyA), FTGAIGGIIAMAITGGIF (SEQ ID NO: 3) (YaxA), or LVDAFKDLIPTGENLSELDLAKPEIELLKQSLEITKKLLGQF (SEQ ID NO: 4) (YaxB).

In yet another preferred embodiment, the alpha-helical TM sequence of the decameric pore of AhlB: Aeromonas hydrophila is used. (PDB: 6GRJ; Wilson et al. Nat Commun, 10:2900-2900, 2019).

In a still further aspect, the TM sequence APLVRWNRVISQLVPT ISGVHDMTETVRYIKRWPN (SEQ ID NO: 5) of Wza, an integral outer membrane protein responsible for exporting a capsular polysaccharide in Escherichia coli (PDB: 2J58; Dong et al. (2006) Nature 444: 226) is used.

Alternatively, the artificial nanopore comprises the TM region of a β-barrel pore forming protein or β-PFPs, which are so-named because they are composed mostly of β-strand-based domains. They have divergent sequences, and are classified by Pfam into a number of families including Leukocidins, Etx-Mtx2, Toxin-10, and aegerolysin. X-ray crystallographic structures have revealed some commonalities: α-hemolysin and Panton-Valentine leukocidin S are structurally related. Similarly, aerolysin and Clostridial Epsilon-toxin and Mtx2 are linked in the Etx/Mtx2 family. In a preferred embodiment, a nanopore of the present invention comprises the TM region of α-heamolysin, aerolysin or anthrax protective antigen (PA).

In a specific aspect, the TM sequence comprises or consists of the amino acid sequence VHGNAEVHASFFDIGGSVSAGF (SEQ ID NO: 6).

### Ring-Forming Protein

An artificial nanopore provided herein is among others characterized by a ring-forming protein that can control the transport of a polymer, e.g. a polypeptide or DNA molecule, across the TM region of the nanopore. For example, it is a toroidal or donut-shaped multi-subunit protein that can dock onto the alpha ring of the 20S proteasome. In one embodiment, it is a ring-forming multimeric protein, such as an octameric, heptameric or hexameric protein. In one aspect, the stoichiometry of the ring-forming multimeric protein is the same as the stoichiometry of that of the pore forming protein from which the TM sequence is derived. For example, the TM region of anthrax protective antigen is suitably combined with a transporting protein forming a heptameric ring. On the other hand, a matching stoichiometry is not essential since many nanopores can assemble with different stoichiometries. For example, a nanopore of the invention may also be based on a soluble protein that is a heptamer and wherein the transmembrane part comes from a hexamer, octamer, nanomer or decamer.

In one embodiment, the ring-forming protein is a heptameric protein that controls or is capable of controlling the transport of a polynucleotide across the TM region. Suitable heptameric proteins include those submitted to the Protein Data Bank (PDB) under one of the following unique accession or identification code codes: 1g31, 1h64, 1hx5, 1i4k, 1i5l, 1i8f, 1i81, 1iok, 1j2p, 1jri, 1lep, 1lnx, 1loj, 1mgq, 1n9s, 1ny6, 1p3h, 1tzo, 1wnr, 1xck, 2cb4, 2cby, 2yf2, 3bpd, 3cf0, 3j83, 3ktj, 3m0e, 3st9, 4b0f, 4emg, 4gm2, 4hnk, 4hw9, 4jcq, 4ki8, 4owk, 4qhs, 4xq3, 5jzh, 5msj, 5msk, 5mx5 and 5uw8e.

Good results can be obtained using a heptameric ATPase protein, preferably A. aeolicus ATPase or a homolog or functional equivalent thereof. For example, the TM sequence of the anthrax protective antigen was fused (by insertion replacement) to a monomer of Aquifex aeolicus ATPase, which functions as a molecular motor to permit DNA melting and stabilization of open complexes (FIG. 9A, FIG. 9B, FIG. 9C, and FIG. 9D).

In another embodiment, the ring-forming multimeric protein is a heptameric protein that controls or is capable of controlling the transport of a polypeptide across the TM region. Very good results are obtained with subunits of the heptameric mammalian proteasome activator PA28 or a homolog or functional equivalent thereof (see Examples 1-5). The heptameric proteasome activator (PA) 28αβ is known to modulate class I antigen processing by docking onto 20S proteasome core particles (CPs) (see Huber et al. Structure. 2017 Oct. 3; 25(10):1473-1480). In one aspect, the PA28alpha subunit or a homolog thereof is used (See Examples 1-4). In another embodiment, the PA28beta subunit or a homolog thereof is used. In a still further embodiment, the PA28gamma subunit or a homolog thereof is used.

PA28 homologs can be derived from the art. Alignment of mouse PA28 sequences responsible for proteasome binding (activation loop and C termini) revealed key sequences in the regions 143-149 and 241-249. Homologous sequences can be found in other sequences, such as the PA26 subunit from Trypanosoma brucei. (see PA26: The 1.9 Å structure of a proteasome-11S activator complex and implications for proteasome-PAN/PA700 interactions. Mol. Cell 18, 589-599 (2005)). In a specific aspect, the invention provides an artificial PA26-nanopore (see Example 5). An artificial nanopore according to the invention can be considered to comprise a hydrophobic part represented by the transmembrane, pore-forming region, fused to a water-soluble part represented by the ring-forming protein that controls the translocation of a substrate (e.g. polypeptide or polynucleotide) across the pore. To that end, a TM amino acid sequence of a β-barrel or α-helical pore forming protein is fused to an amino acid sequence of (ii) a subunit of a ring-forming multimeric protein capable of controlling the transport of a polypeptide or polynucleotide across the TM region of the assembly. The amino acids that are present at the “fusion interface” between the two parts are thought to be in contact with the hydrophobic membrane and the hydrophilic layer that keeps the membrane hydrated (e.g. the phosphate group in phospholipids), and of relevance for insertion efficiency and nanopore stability. In one embodiment, the TM sequence is N-or C-terminally fused to the subunit of a ring-forming multimeric protein. In another embodiment, the TM sequence is inserted within the sequence of the subunit of a ring-forming protein. In some cases, it is desirable to remove one or more residues from the native sequence of a subunit of a ring-forming multimeric protein to optimize nanopore formation. Thus, as used herein, the expression “wherein the TM sequence of a β-barrel or α-helical pore forming protein is fused to the amino acid sequence of a subunit of a ring-forming (multimeric) protein” encompasses (i) genetic fusion of a TM sequence to either the (optionally truncated) N-or C-terminus of a ring forming protein subunit; (ii) insertion of a TM sequence within the sequence of a ring forming protein subunit; and (iii) insertion of a TM sequence concomitant with a deletion of a sequence of a ring forming protein subunit. In the latter case, the size of the deleted sequence can be smaller, larger or identical to that of the inserted TM sequence. In all three cases, the TM sequence may be flanked at the fusion site(s) with a flexible linker.

The site of insertion, replacement or addition of the TM sequence can vary depending on the protein used, but it is typically made by replacing a loop in the ring-forming protein that is located perpendicularly to the lipid bilayer and parallel to the opening of the newly formed artificial nanopore. The loop can be from a few to tens of amino acids long. Typically, the loop to be deleted contains one or more disordered regions. In one aspect, insertion is accompanied by replacing (exchanging) a stretch of amino acids of the ring-forming protein. For example, very good results can be achieved when a TM sequence is inserted in an AP28 subunit while replacing its so-called “disorder region”, represented by the amino acid residues 63-100 of AP28. As another example, a TM sequence is inserted in a subunit of an ATPase of A. aeolicus while replacing a stretch of nine amino acid residues of the ATPase subunit.

Alternatively, the N-or the C-terminus of the ring-forming protein can be replaced or extended by a TM sequence that will form a transmembrane region.

### Flexible Linkers

To allow for optimal function (e.g. membrane insertion, bilayer stability), the inserted TM sequence may (yet does not need to) be flanked on the N-and/or C-terminal side by a flexible hydrophilic linker of at least 3 amino acids, preferably at least 5 amino acids, e.g. 5-20 amino acids. As used herein, the term “hydrophilic” refers to amino acids whose side chains can interact with the charged head groups of membrane (phospho)lipids. For example, hydrophilic residues include serine, threonine, asparagine, glutamine, aspartate, glutamate, lysine and arginine. In many examples found in nature, amphipathic-hydrophobic residues (tyrosine, tryptophan and histidine) mediate the interaction between the protein and the lipid bilayer and these can therefore also be used.

In one embodiment, at least 50% of the amino acids of the flexible hydrophilic linkers are Ser and/or Thr residues. Possibly, at least 50% of the amino acids are Ser residues. The flexible linkers flanking the C-and N-terminal sides of the TM spanning domain can have the same or a distinct (e.g. inverted) sequence. For example, the N-terminal linker comprises or consists of the sequence GSS, whereas the C-terminal linker consists of the sequence SSG.

The invention herewith provides a generic method to insert a protein with toroidal structure into a lipid bilayer. In order to study the effect of the linker chemical composition on the electrical property of the nanopore, we screened several different hydrophilic amino acids. The length of linkers on the N-terminal side (β1) and C-terminal side (β2) was kept fixed to 5 residues. β1 appeared to tolerate most of mutations. By contrast, even small changes to β2 increased the noise of electrical recordings at both potentials (data not shown). Interestingly, however, a construct in which all the five amino acids in both linkers were substituted to serine showed high stability and formed nanopores with homogenous unitary currents.

### Fusion to Proteasome Alpha-Subunit

In order to allow for the application of an artificial nanopore of the invention for single-molecule protein analysis, it is advantageously connected hermetically (i.e. by genetic fusion) to the 20S proteasome, in particular to the alpha-subunit thereof. Advantageously, the S20 proteasome from Thermoplasma acidophilum is used, which is a multi-subunit protease that degrades polypeptides at physiological conditions and also extreme conditions (high salt, high temperature and low pH).

In one embodiment, the invention provides an artificial nanopore as described herein above, wherein the C-terminus of a subunit of the ring-forming (multimeric) protein comprising (by insertion replacement) the flanked TM sequence is genetically fused to the N-terminus of a proteasome α-subunit. Preferably, it is fused to an N-terminally truncated proteasome α-subunit such that the proteasome gate is left open towards the nanopore. In one embodiment, the proteasome α-subunit lacks the at least 15 N-terminal amino acids (e.g. residues 1-15, 1-17, 1-19, 1-20, 1-21, 1-22 or 1-25). Preferably, at least 20 N-terminal residues are removed (αΔ20). For example, the C-terminus of the ring-forming multimeric protein comprising the flanked TM region is genetically fused to residue L21 of the proteasome α-subunit. Deletion of more than about 30 residues is not recommended to safeguard proteasome function.

In a specific aspect, the invention provides an artificial nanopore wherein the C-terminus of PA28 comprising the flanked TM region of anthrax protective antigen (PA) is genetically fused to the N-terminus of a proteasome α-subunit, preferably αΔ20, more preferably T. acidophilum αΔ20.

### Fusion to Clp Protease-Subunit

In another embodiment, order to allow for the application of an artificial nanopore of the invention for single-molecule protein analysis, it is advantageously connected hermetically (i.e. by genetic fusion) to a member of the Clp protease (ClpP) family.

The Clp protease family contains serine peptidases that belong to the MEROPS peptidase family S14 (ClpP endopeptidase family, clan SK). ClpP is an ATP-dependent protease that cleaves a number of proteins, such as casein and albumin. It exists as a heterodimer of ATP-binding regulatory A and catalytic P subunits, both of which are required for effective levels of protease activity in the presence of ATP, although the P subunit alone does possess some catalytic activity.

Proteases highly similar to ClpP have been found to be encoded in the genome of bacteria, metazoa, some viruses and in the chloroplast of plants. A number of the proteins in this family are classified as non-peptidase homologues as they have been found experimentally to be without peptidase activity, or lack amino acid residues that are believed to be essential for catalytic activity.

As is demonstrated herein below, an artificial nanopore capable of single protein analysis was obtained when the N-terminus of a subunit of the ring-forming multimeric protein comprising a TM sequence was genetically fused to the C-terminus of an Clp protease (ClpP) subunit. More in particular, the invention provides an artificial nanopore based on an artificial PA28-nanopore as described herein above, wherein a subunit of ClpP (PDB ID: 1TYF) is fused at the N-terminus of PA28-nanopore (see Example 7).

### Multi-Protein Nanopore Sensor Assembly/Complex

A further aspect relates to a stable multi-protein assembly or subcomplex comprising components of the 20S proteasome, which subcomplex can function as an artificial transmembrane proteasome. The 20S proteasome from Thermoplasma acidophilum has a cylindrical structure made of four stacked rings composed of 14 α- and 14 β-subunits (FIG. 1E)¹². The two flanking outer α-rings allow for the association of the 20S proteasome with several regulatory complexes13, among which is proteasome activator PA28 (FIG. 1A) that controls the translocation of substrates into the catalytic cavity¹⁴.

In one embodiment, the invention provides a multi-protein nanopore sensor assembly/complex, comprising (i) an artificial nanopore as described herein above, together with (ii) a ring composed of proteasome α-subunits and optionally (iii) a ring composed of proteasome β-subunits wherein (ii) and (iii) are present as separate proteinaceous components i.e. not fused or otherwise connected to the nanopore. In one embodiment, a multi-protein complex comprises an artificial nanopore that is complexed to a “free” ring of proteasome α-subunits. For example, this design is suitably used for translocating polypeptides at a controlled speed without the need to process them by the proteasomal peptidase.

Preferably, the invention provides a multi-protein nanopore sensor assembly/complex, comprising (i) an artificial nanopore as described herein above, together with (ii) one or two rings composed of proteasome α-subunits and optionally (iii) one or two rings composed of proteasome β-subunits. Such complex is herein also referred to as “transmembrane proteasome” or “proteasome nanopore”. For example, the complex may comprise (i) an artificial nanopore (e.g. TM-PA28-α-subunit) (ii) one ring composed of proteasome α-subunits and (iii) two rings composed of proteasome β-subunits.

The N-terminus of the proteasome α-subunit comprised in a multi-protein assembly may be truncated in order to allow for a fast degradation of unfolded protein substrates without the need for a proteasome activator. For example, a proteasome α-subunit lacking the at least 5, preferably at least 10, more preferably at least 12 N-terminal amino acids is used.

The proteasome β-subunit may be used as such in a multi-protein assembly. The three naturally occurring β-type subunits contain catalytically active threonine residues at their N termini and show N-terminal nucleophile (Ntn) hydrolase activity, indicating that the proteasome is a threonine protease that does not fall into the known seryl, thiol, carboxyl and metalloprotease families. The βsubunits are associated with caspase-like/PGPH (peptidylglutamyl-peptide hydrolyzing), trypsin-like and chymotrypsin-like activities, respectively, which confer the ability to cleave peptide bonds at the C-terminal side of acidic, basic and hydrophobic amino-acid residues, respectively.

Alternatively, the complex comprises a ring of proteasome β-subunits that are engineered to provide a different type of protease activity, allowing for a distinct substrate specificity. For example, the modified proteasome β-subunit may have a trypsin-type or chymotrypsin-type of activity. See for example: Ma et al., (2005). Specificity of trypsin and chymotrypsin: loop-motion-controlled dynamic correlation as a determinant. Biophysical J. 89(2), 1183-1193), showing that the activity of trypsin can be converted to chymotrypsin-like protease by replacing the two loops of trypsin with those of chymotrypsin.

The complex may further comprise a protein translocase which can bind, unfold, and translocate a polynucleotide or polypeptide through the nanopore sensor complex in sequential order. For example, the protein translocase is an NTP-driven unfoldase, preferably an AAA+ unfoldase. See for example US2016/0032235 and Dougan et al . (FEBS Letters 529 (2002) 1873-3468).

Members of the AAA+ superfamily have been identified in all organisms studied to date. They are involved in a wide range of cellular events. In bacteria, representatives of this superfamily are involved in functions as diverse as transcription and protein degradation and play an important role in the protein quality control network. Often, they employ a common mechanism to mediate an ATP-dependent unfolding/disassembly of protein-protein or DNA-protein complexes. In an increasing number of examples it appears that the activities of these AAA+ proteins may be modulated by a group of otherwise unrelated proteins, called adaptor proteins.

For example, a complex of the invention comprises the prokaryotic AAA+ unfoldase ClpX. ClpX unfolds substrate proteins by ATP-driven translocation of the polypeptide chain through the central pore of its hexameric assembly. In complex with the ClpP peptidase, ClpX carries out protein degradation by translocating unfolded substrates directly into the ClpP proteolytic chamber (Sauer et al., 2004). In a specific aspect, the invention provides a multi-protein nanopore sensor complex comprising an artificial ClpP nanopore, e.g. by fusion to PA, which sensor complex further comprises ClpX or a homologous protein unfoldase. See Example 7 herein below.

In another embodiment, the protein translocase is the Thermoplasma VCP-like ATPase from Thermoplasma acidophilum (VAT), a member of the two-domain AAA ATPases and homologous to the mammalian p97/VCP and NSF proteins. In another embodiment, the proteasome-activating nucleotidase (PAN) from Methanococcus jannaschii is used, which is a complex of relative molecular mass 650,000 that is homologous to the ATPases in the eukaryotic 26S proteasome. Other examples include AMA, an AAA protein from Archaeoglobus and methanogenic archaea. In a still further embodiment, the translocase is the open reading frame number 854 in the M. mazei genome (Forouzan, Dara, et al. “The archaeal proteasome is regulated by a network of AAA ATPases.” J. Biological Chemistry 287.46 (2012):39254-39262).

Other suitable translocases for use in the present invention include MBA (membrane-bound AAA; Serek-Heuberger, Justyna, et al. “Two unique membrane-bound AAA proteins from Sulfolobus solfataricus.” (2009):118-122) and SAMPs (Humbard, Matthew A., et al. “Ubiquitin-like small archaeal modifier proteins (SAMPs) in Haloferax volcanii.” Nature 463.7277 (2010):54).

Preferred polynucleotide translocases include helicases (e.g. gp4), exonucleases (lambda exonuclease), proteases translocases (e.g. Ftsk), and topoisomerases (e.g. topoisomerase II).

As is exemplified herein below, a transmembrane proteasome inserted efficiently in lipid bilayers and showed low-noise current recordings. Activity assays revealed that the proteasome nanopore was active, with the proteolytic activity increasing with the temperature and decreasing with the salt concentration. The current-voltage (I-V) curve of the proteasome-nanopore in 1 M NaCl solutions was similar to that of PA-nanopore, suggesting that the transmembrane region was unchanged and the gate of the α-subunit was open. A further aspect of the invention therefore relates to an analytical system comprising an artificial nanopore or a multiprotein nanopore complex according to the invention. Typically, by virtue of its TM region, the nanopore is inserted in a hydrophobic membrane that separates a fluid chamber of said system into a cis side and a trans side. For example, the membrane can be a lipid bilayer or it can be a non-lipid system, such as a block copolymer or other type of artificial membrane.

Also provided is a method for translocating a polynucleotide or polypeptide through an analytical system according to the invention. In a specific aspect, the invention relates to a method for single molecule analysis, preferably for identification and/or sequencing of a biopolymer, more preferably for single molecule polypeptide or polynucleotide sequencing, comprising adding a biopolymer to be analyzed to the chamber of an analytical system such that the biopolymer can contact and access the (proteasome) nanopore.

Depending on the conditions used, e.g. ATP concentration, buffer types, the type of analysis can be selected according to needs. For example, VAT is capable of feeding the polypeptide through the nanopore at a speed that can be tuned by the concentration of ATP. We show that the transmembrane proteasome is capable of simultaneously processing and identifying different protein substrates (FIG. 1H). In one embodiment, the system is therefore used in the so-called “degradation mode” wherein translocated peptides are proteolytically degraded. Alternatively, an inactivated proteasome recognizes proteins as they are linearized and transported across the nanopore at a controlled speed (FIG. 1I). This system allows monitoring the activity of the proteasome at the single molecule level, and has applications e.g. in real-time protein sequencing applications. Hence, in another embodiment, the system is used in the so-called “translocation mode”.

Also provided herein is the use of a system comprising an artificial nanopore or a multiprotein nanopore complex according to the invention for single molecule analysis, preferably for identification and/or sequencing of a biopolymer, more preferably for single molecule polypeptide or polynucleotide sequencing. We envisage two ways to sequence proteins. In the (active) peptide-mode the proteasome will recognize a protein, cut it into pieces and recognize the individual fragments. In the inactive strand-mode, proteins can be recognized as they are linearized and transported across the nanopore at a controlled speed by unfoldase, for example VAT, which threads intact substrates across the nanopore channel. Individual peptides are directed by the electroosmotic flow through the proteasomal nanochannel to the nanopore where they are recognized by specific current blockades. Herewith, the invention provides a multi-protein proteasome-nanopore for real-time single-molecule protein sequencing applications. It is the first multicomponent proteolytic nanopore that controls the transport of polypeptides across a nanopore. Notably, the proteasome-nanopore degrades polypeptides not only at physiological conditions, but also under more extreme conditions including high salt, high temperature and/or low pH. Importantly, it is shown that proteins can also be discriminated under the above mentioned conditions.

The invention also provides means and methods for providing an artificial nanopore of the invention. In one embodiment, it provides a nucleic acid molecule encoding a subunit of an artificial nanopore as herein disclosed. The nucleic acid molecule encodes a fusion protein comprising (i) the transmembrane (TM) sequence of a β-barrel or α-helical pore forming protein fused to the amino acid sequence of (ii) a subunit of a ring-forming (multimeric) protein capable of controlling the transport of a polypeptide or polynucleotide across the TM region of the assembly.

In one embodiment, the nucleic acid molecule encodes a fusion protein comprising (i) the TM sequence of a β-barrel or α-helical pore forming protein flanked on the N-and C-terminal side by (ii) a flexible linker of at least 3 amino acids, the flanked TM sequence being inserted in the amino acid sequence of (iii) a subunit of a ring-forming (multimeric) protein capable of controlling the transport of a polypeptide or polynucleotide across the TM region. In a preferred embodiment, the nucleic acid molecule encodes the above fusion protein wherein the C-terminus of the ring-forming multimeric protein comprising the flanked TM sequence is genetically fused to the N-terminus of a proteasome α-subunit, optionally lacking the at least 15 N-terminal amino acids. In another preferred embodiment, the nucleic acid molecule encodes the above fusion protein wherein the N-terminus of the ring-forming multimeric protein comprising the flanked TM sequence is genetically fused to the C-terminus of a subunit of a ClpP family member.

Other nucleic acid molecules for use in the invention may encode a (N-terminally truncated) proteasomal α-subunit or a proteasomal β-subunit. Any protein encoded by a nucleic acid molecule of the invention may comprise, e.g. at its N-or C-terminus, a protein tag allowing for purification and/or isolation of the protein. For example, a His-tag or Strep-tag can be added. Other preferred nucleic acids molecules include those encoding the preferred artificial nanopores as described herein above.

Also provided is an expression vector comprising a nucleic acid molecule according to the invention, and a host cell e.g. bacterial or yeast host cell, comprising the expression vector. The host cell may further comprise (i.e. be co-transfected with) a distinct expression vector encoding a proteasome beta-subunit and/or a proteasome alpha-subunit. In a specific aspect, a host cell comprises two separate vectors, one of which encodes a (His-tagged) artificial nanopore subunit fused a proteasomal α-subunit, and the other encodes a proteasomal β-subunit and a second (Strep-tagged) proteasomal α-subunit. Expression of such host cell allows for the recombinant production and co-assembly of all components of a multi-protein artificial proteasome-nanopore complex. Proteins can be isolated according to methods known in the art, for example using affinity chromatography exploiting the presence of one or more protein tag(s) and/or co-purification based on the natural affinity of the proteins for each other. See in particular FIG. 4B.

### BRIEF DESCRIPTION OF THE DRAWINGS

Design of a transmembrane protein device for single-molecule protein analysis. FIG. 1A, Structure of mouse PA28α (PDB ID: 5MSJ). FIG. 1B, Sticks diagram of the structure of serine-serine-glycine linker. FIG. 1C, Ribbon diagram of the structure of anthrax protective antigen (PDB ID: 3J9C). The transmembrane region of the protective antigen is in magenta. The lipid molecules are indicated schematically by a circular polar head region and two flexible acyl chains. FIG. 1D, Structure of artificial nanopore generated by molecular dynamics simulations. PA28 (FIG. 1A) was genetically fused to the transmembrane region of the protective antigen (FIG. 1C) via a short linker (FIG. 1B). FIG. 1E, Structure of T. acidophilum proteasome α and β subunit (PDB ID: 1YA7). FIG. 1F, Structure of the designed proteasome nanopore. FIG. 1G, Structure of the Thermoplasma VCP-like ATPase from Thermoplasma acidophilum (VAT) (PDB ID: 5G4G), FIG. 1H and FIG. 1I, VAT bound to the artificial nanopore. Then the translocated protein is degraded to peptides (FIG. 1H) or released (FIG. 1I).

Fabrication and electrical optimization of a nanopore. FIG. 2A, Effects of linker length on the nanopore expression in E. coli cells, insertion efficiency and nanopore stability. The transmembrane region was inserted in the middle of PA28 via a short linker (SSG, red). Three phenylalanine and one valine residue define the lipid-water boundary, and are highlighted with green squares. The side chains that point towards the outside and inside of the barrel are highlighted with gray and black lines, respectively. Each of the seven subunits contributes two β-strands separated by a turn (black line). The firstly designed nanopore is highlighted with wider arrow. One deletion mutant (Δ2) and five insertion mutants (∇2, ∇4, ∇8, ∇12, and ∇16) were prepared based on the native sequence of the protective antigen. For the sake of clarity, PA28 is shown as a cyan square. FIG. 2A discloses SEQ ID NOS 27-28, and 38, respectively, in order of appearance. FIG. 2B, Electrical properties of ∇4 mutant. Left: the linker sequence of ∇4 mutant. Middle: electrical recordings of a single nanopore at ±35 mV. Right: Histogram of the unitary conductance values of 59 nanopores at −35 mV. FIG. 2B discloses SEQ ID NOS 29-30, respectively, in order of appearance. FIG. 2C, Electrical properties of ∇2 mutant. Left: the linker sequence of ∇2 mutant. Middle: Typical current trace and the current histogram corresponding the insertion of individual pore into a lipid membrane at +35 mV. Right: Histogram of the unitary conductance values of 59 artificial nanopores at −35 mV. Data were collected at ±35 mV in 1 M NaCl, 15 mM Tris, pH 7.5 using 10 kHz sampling rate and a 2 kHz low-pass Bessel filter. FIG. 2C discloses SEQ ID NOS 31-32, respectively, in order of appearance. FIG. 2D, Interaction of DPhPC with the artificial transmembrane pore generated by molecular dynamics simulations.

Electrical properties of optimized artificial pore (∇2) and discrimination of substrates. FIG. 3A, Schematic of the ion-current measurement setup. The artificial pore is added to the cis side, and inserted into a suspended lipid membrane. An electrical potential is applied via two Ag/AgCl electrodes, which induces a current of Na⁺ and Cl⁻ ions through the nanopore (1 M NaCl, 15 mM Tris, pH 7.5). The pore is colored blue (positive) and red (negative) according to the vacuum electrostatic potential as calculated by PyMOL. FIG. 3B, A typical current trace recorded through an efficient single pore after optimization at ±35 mV. The average current value is 41.24±0.02 pA at −35 mV and 45.43±0.06 pA at +35 mV. FIG. 3C, Averaged current-voltage (I-V) characteristics of three different nanopores. The error bars represent a standard deviation from the mean curve. FIG. 3D, Ion selectivity of the nanopore. Determination of the reversal potential shows that the pore is cation-selective, as expected from the electrostatic potentials at their constrictions (FIG. 3A). The current signals were filtered at 2 kHz and sampled at 10 kHz. FIG. 3E, Chemical structure of β-CD, scatter plots of Iᵣₑₛ % versus dwell time, and representative trace. FIG. 3F, Chemical structure of γ-CD, scatter plots of Iᵣₑₛ % versus dwell time, and representative trace. FIG. 3G, Peptide sequences of angiotensin I, scatter plots of Iᵣₑₛ % versus dwell time, and representative trace. FIG. 3G discloses SEQ ID NO: 33. FIG. 3H, Peptide sequences of dynorphin A, scatter plots of Iᵣₑₛ % versus dwell time, and representative trace. FIG. 3H discloses SEQ ID NO: 34.

Design of the artificial proteasome-nanopore. FIG. 4A, Structure of T acidophilum proteasome-PA26. PA26, proteasome α subunit, and β subunit are colored orange/magenta, and green, respectively. The C-terminal of PA26 (S231) is near L21 of the α subunit. FIG. 4B, Reconstitution of artificial proteasome-nanopore. To obtain subcomplex 3, two separate vectors were used to express the four proteins. PA pore was fused to the proteasome α subunit (αΔ20) with the N-terminal His-tag and cloned into pET-28a vector. Untagged β subunits and a second α subunit (αΔ12) with the C-terminal Strep-tag were cloned into pETDuet-1 vector. First a His-tag affinity chromatography co-purified complex 1 and 3. Then a Strep-Tag affinity chromatography purified 3. FIG. 4C, SDS-PAGE (left) and native PAGE (right) analyses of the purified complex 3. SDS-PAGE revealed the presence of three unique bands of PAαΔ20 (Top), αΔ12 (middle), and β (bottom) with molecular weights of 52.7, 25.8, and 22.3 kDa, respectively. These results suggest that PAαΔ20, β, and αΔ12 form a stable subcomplex 3. The native PAGE showed only one band indicating that the complex is stable. FIG. 4D, Behavior of a single pore at ±35 mV in 1 M NaCl, 15 mM Tris, pH 7.5. Subcomplex 3 displayed some fast gating behavior at positive potential. FIG. 4E, Cut-through of a surface representation of artificial transmembrane proteasome colored (blue, positive; red, negative) according to the vacuum electrostatic potential as calculated by PyMOL.

SDS-PAGE analysis the hydrolyzing activity of subcomplex 3. FIG. 5A, β-casein (1 mg/mL) was incubated with subcomplex 3 at 53° C. in buffer A (50 mM Tris, pH 7.5, 150 mM NaCl). FIG. 5B, β-casein (1 mg/mL) was incubated with subcomplex 3 for 2 hours in buffer A. FIG. 5C, β-casein (1 mg/mL) was incubated with subcomplex 3 at 53° C. for 0.5 hour in buffer B (50 mM Tris, pH 7.5, 0.3-1.0 M NaCl). The β-casein/subcomplex 3 concentration ratio was 42.

Discrimination of substrates with the proteasomal nanopore. FIG. 6A, Typical current trace provoked by substrate 1 (S1) using an inactive proteasome-nanopore. FIG. 6B, Translocation of S1 (20 μM) through an inactive proteasome-nanopore mediated by VAT (20.0 μM) and ATP (2.0 mM). FIG. 6C, When an inactive proteasome is used in the presence of ATP and VAT, GFP-ssrA is unfolded and translocated intact through the proteasome chamber and nanopore. FIG. 6D, Typical current traces provoked by Si using an active proteasome-nanopore. FIG. 6E, When an active proteasome is used, in the presence of VAT and ATP, only rare and fast events are observed suggesting that the active proteasome-nanopore cleaves S1 efficiently producing small fragments. FIG. 6F, When an active proteasome is used in the presence of ATP and VAT, unfolded GFP-ssrA is cleaved in the proteasomal chamber and the degraded peptides are too short to be detected by the nanopore. Data were collected at 40° C. and −30 mV in 1 M NaCl, 15 mM Tris, pH 7.5.

Discrimination of substrates with proteasomal nanopore. FIG. 7A, Sequence comparison of substrate 1 and 2. FIG. 7B, Scatter plots of fraction blockade versus time and representative blockades induced by cleaved S1 and S2 at 40° C. and −30 mV in 1 M NaCl, 15 mM Tris, pH 7.5. FIG. 7A discloses SEQ ID NOS 39-40, respectively, in order of appearance. “PVPLPIP” disclosed as SEQ ID NO: 35, and “PVPLPIPVPLPIPVPLPIP” disclosed as SEQ ID NO: 36.

Design and membrane insertion of PA26 artificial nanopore. FIG. 8A, Ribbon diagram of the structure of anthrax protective antigen (PDB ID: 3J9C). The transmembrane region is highlighted in blue. FIG. 8B, Structure of PA26 (PDB ID: 1YA7). FIG. 8C, Structure of artificial PA26-nanopore. FIG. 8D, Typical current trace shows insertion of individual pore. Data were collected at ±35 mV in 1 M NaCl, 15 mM Tris, 20 mM MgCl₂, pH 7.5.

Design and insertion of ATPase artificial nanopore. FIG. 9A, Ribbon diagram of the structure of anthrax protective antigen (PDB ID: 3J9C). The transmembrane region is highlighted in blue. FIG. 9B, Structure of Aquifex aeolicus ATPase (PDB ID: 3M0E). FIG. 9C, Structure of artificial ATPase transmembrane pore. FIG. 9D, Typical current trace shows insertion and ATP hydrolysis of individual pore. The ATPase nanopore displayed gating at positive potentials. The current traces became noisy and bigger when ATP (2 mM) was added in solution. Data were collected at ±35 mV in 1 M NaCl, 15 mM Tris, 20 mM MgCl₂, pH 7.5.

Design of a ClpP-artificial nanopore for single-molecule protein analysis. FIG. 10A, Structure of PA-nanopore. FIG. 10B and FIG. 10C, Ribbon diagram of the structure of ClpP (PDB ID: 1TYF). FIG. 10D, PA-nanopore was genetically fused to ClpP. FIG. 10E, Structure of the designed ClpP-nanopore. FIG. 10F, Structure of unfoldase ClpX (PDB ID: 3HWS).

FIG. 11|Current-voltage (I-V) characteristics of three different nanopores. The artificial opened and closed ClpP-nanopore did not alter the conductance of the nanopore. The current signals were recorded in 0.5 M KCl, 20 mM HEPES, pH 7.5, filtered at 2 kHz, and sampled at 10 kHz.

FIG. 12|Controlled translocation through the ClpP-nanopore. ClpX assisted transport of GFP across opened ClpP-nanopore in the presence of 2.0 mM ATP. The ClpP-nanopore, ClpX and GFP were added to the cis side. Data were collected at 22° C. and −50 mV in 0.1 M KCl, 0.3 M NaCl, 10% glycerol, 15 mM Tris, pH 7.5, using a 10 kHz low-pass Bessel filter with a 50 kHz sampling rate. The traces were then filtered digitally with a Gaussian low-pass filter with a 5 kHz cut-off.

### EXPERIMENTAL SECTION

### Materials and Methods

General materials. Oligonucleotides and gBlock gene fragments were obtained from Integrated DNA Technologies (IDT). Phire Hot Start II DNA Polymerase, restriction enzymes, T4 DNA ligase, and Dpn I were purchased from Fisher Scientific. Angiotensin I, dynorphin A, pentane, hexadecane, and Trizma base were obtained from Sigma-Aldrich. 1,2-diphytanoyl-sn-glycero-3-phosphocholine (DPhPC) was purchased from Avanti Polar Lipids. Sodium chloride and Triton X-100 was bought from Carl Roth.

Plasmid Construction for proteins. gBlock gene fragments were ordered for synthesis by IDT, and cloned into pT7-SC1 plasmid³³ using Nco I and Hind III restriction digestion sites. Plasmid and gene were ligated together using T4 ligase (Fermentas). 0.5 μL of the ligation mixture was incorporated into 50 μL E. cloni® 10G (Lucigen) competent cells by electroporation. Transformants were grown overnight at 37° C. on LB agar plates supplemented with ampicillin (100 μg/mL). Ampicillin-resistant colonies were picked and inoculated into 5 mL LB medium supplemented with of ampicillin (100 μg/mL) for plasmid DNA preparation. The plasmid was extracted with GeneJET Extraction Kit (Fisher Scientific). The identity of the clones was confirmed by sequencing at Macrogen.

Plasmid Construction for building a sequencing proteasome machine. gBlock gene fragments of Thermoplasma acidophilum α and β were ordered for synthesis by IDT. The gene encoding for the α subunit was cloned upstream of pETDuet-1 vector (Novagen) between the Nco I and Hind III sites with the gene of Strep-tag at the C-terminus. Subsequently, the gene encoding for an untagged β subunit was cloned downstream between the Nde I and Kpn I sites. PA-nanopore was fused to α subunit gene through PCR splicing by overlap extension³⁴, and cloned into pET-28a vector (Novagen) using Nco I and Hind III restriction digestion sites with His tag at the N terminus.

Construction of mutants. All mutants were constructed using the QuickChange protocol³⁵ for site-directed mutagenesis on a circular plasmid template DNA with Phire Hot Start II Polymerase. Partially overlapping primers were used to avoid primer self-extension. PCR amplification was as follows: denaturation at 98° C. for 3 min, followed by 30 cycles of 98° C. for 30 s, 55° C. for 30 s, and 72° C. for 3 min, and a final extension cycle of 72° C. for 5 min. After the PCR reaction, the parental DNA template was digested with Dpn I enzyme for 1 h at 37° C. The PCR amplified plasmid was separated on 1% agarose gel, extracted with GeneJET Gel Extraction Kit (Fisher Scientific), and incorporated into 50 μL E. cloni® 10G (Lucigen) competent cells by electroporation. Transformants containing the plasmid were grown overnight at 37° C. on LB agar plates supplemented with ampicillin (100 μg/mL). Ampicillin-resistant colonies were picked and inoculated into 5 mL LB medium supplemented with of ampicillin (100 μg/mL) for plasmid DNA preparation. The plasmid was extracted with GeneJET Extraction Kit (Fisher Scientific), and sequenced at Macrogen for confirmation of the mutation.

Expression and purification. The gene of the PA nanopore was transformed into E. coli. BL21 (DE3) pLysS chemically competent cells. Transformants were selected after overnight growth at 37° C. on lysogeny broth (LB) agar plates supplemented with ampicillin (100 mg/L). The resulting colonies were inoculated into 200 mL LB medium containing 100 mg/L of ampicillin. The cells were grown at 37° C. (180 rpm shaking). After the optical density reached an absorbance of 0.6 at 600 nm, the expression was induced by addition of 0.5 mM isopropyl β-D-1-thiogalactopyranoside (IPTG). The temperature was lowered to 25° C., and the cell cultures were further grown overnight. The cells were harvested by centrifugation for 20 min (4000×g) at 4° C. and the pellets were stored at −80° C. About 100 mL of cell culture pellet was thawed and solubilized with ˜20 mL lysis buffer (150 mM NaCl, 50 mM Tris-HCl, pH 7.5, 1 mM MgCl2, 0.1 units/mL DNase I, 10 μg/mL lysozyme, 1% v/v Triton X-100) and stirred with a vortex shaker for 1 hour at 22° C. The bacteria were then lysed by sonication (duty cycle 10%, output control 3, Branson Sonifier 450). The lysate was subsequently centrifuged at 6000×g at 4° C. for 20 min and the cellular debris discarded. The supernatant was mixed with 100 μL of Strep-Tactin resin (IBA) to a 50 mL falcon tube, which was pre-equilibrated with wash buffer (1% v/v Triton X-100, 150 mM NaCl, 15 mM Tris-HCl, pH 7.5). After 1 hour, the resin was loaded into a column (Micro Bio Spin, Bio-Rad), which was pre-washed with 5 mL wash buffer (150 mM NaCl, 50 mM Tris-HCl, pH 7.5, 1% v/v Triton X-100). In total, 10 mL of wash buffer (1% v/v Triton X-100, 150 mM NaCl, 50 mM Tris, pH 7.5, 20 mM imidazole) was used to wash the beads. The protein was eluted with approximately 100 μL elution buffer (2.5 mM desthiobiotin, 150 mM NaCl, 50 mM Tris-HCl, pH 7.5, 0.2% v/v Triton X-100).

The genes encoding for test peptides S1 and S2 were separately transformed into E. coli. BL21 (DE3) electrocompetent cells. Transformants were selected after overnight growth at 37° C. on lysogeny broth (LB) agar plates supplemented with ampicillin (100 mg/L). The resulting colonies were inoculated into 200 mL LB medium containing 100 mg/L of ampicillin. The cells were grown at 37° C. (180 rpm shaking). After the optical density reached an absorbance of 0.6 at 600 nm, the expression was induced by addition of 0.5 mM isopropyl β-D-1-thiogalactopyranoside (IPTG) at 37° C. And the cell cultures were further grown 4 hours. The cells were harvested by centrifugation for 20 min (4000×g) at 4° C. and the pellets were stored at −80° C. About 100 mL of cell culture pellet was thawed and solubilized with ˜20 mL lysis buffer (300 mM NaCl, 50 mM Tris-HCl, pH 7.5, 1 mM MgCl2, 0.1 units/mL DNase I, 10 μg/mL lysozyme, 0.2% v/v Triton X-100) and stirred with a vortex shaker for 1 hour at 4° C. The bacteria were then lysed by sonication (duty cycle 10%, output control 3, Branson Sonifier 450). The lysate was subsequently centrifuged at 6000×g at 4° C. for 20 min and the cellular debris discarded. The supernatant was mixed with 100 μL of Ni-NTA resin (Qiagen) to a 50 mL falcon tube, which was pre-equilibrated with wash buffer (300 mM NaCl, 50 mM Tris-HCl, pH 7.5, 0.2% v/v Triton X-100). After 1 hour at 4° C., the resin was loaded into a column (Micro Bio Spin, Bio-Rad), which was pre-washed with 5 mL wash buffer (300 mM NaCl, 50 mM Tris-HCl, pH 7.5, 0.2% v/v Triton X-100). In total, 10 mL of wash buffer (300 mM NaCl, 50 mM Tris, pH 7.5, 20 mM imidazole) was used to wash the beads. The protein was eluted with approximately 200 μL elution buffer (500 mM imidazole, 300 mM NaCl, 50 mM Tris-HCl, pH 7.5).

The genes encoding for VAT and GFP were separately transformed into E. coli. BL21 (DE3) electrocompetent cells. Transformants were selected after overnight growth at 37° C. on lysogeny broth (LB) agar plates supplemented with ampicillin (100 mg/L). The resulting colonies were inoculated into 200 mL LB medium containing 100 mg/L of ampicillin. The cells were grown at 37° C. (180 rpm shaking). After the optical density reached an absorbance of 0.6 at 600 nm, the expression was induced by addition of 0.5 mM isopropyl β-D-1-thiogalactopyranoside (IPTG) at 25° C. And the cell cultures were further grown overnight. The cells were harvested by centrifugation for 20 min (4000×g) at 4° C. and the pellets were stored at −80° C. About 100 mL of cell culture pellet was thawed and solubilized with ˜20 mL lysis buffer (150 mM NaCl, 50 mM Tris-HCl, pH 7.5, 1 mM MgCl2, 0.1 units/mL DNase I, 10 μg/mL lysozyme) and stirred with a vortex shaker for 1 hour at 4° C. The bacteria were then lysed by sonication (duty cycle 10%, output control 3, Branson Sonifier 450). The lysate was subsequently centrifuged at 6000×g at 4° C. for 20 min and the cellular debris discarded. The supernatant was mixed with 100 μL of Ni-NTA resin (Qiagen) to a 50 mL falcon tube, which was pre-equilibrated with wash buffer (150 mM NaCl, 50 mM Tris-HCl, pH 7.5). After 1 hour at 4° C., the resin was loaded into a column (Micro Bio Spin, Bio-Rad), which was pre-washed with 5 mL wash buffer (150 mM NaCl, 50 mM Tris-HCl, pH 7.5). In total, 10 mL of wash buffer (150 mM NaCl, 50 mM Tris, pH 7.5, 20 mM imidazole) was used to wash the beads. The protein was eluted with approximately 200 μL elution buffer (500 mM imidazole, 150 mM NaCl, 50 mM Tris-HCl, pH 7.5).

Proteasome co-expression and purification. For the assembly of the proteasome-nanopore, the pETDuet-1 containing the gene encoding for the α and β subunits of the proteasome and pET28a containing the gene encoding for the PA28-αΔ20 nanopore plasmids were co-transformed into E. coli BL21 (DE3) electrocompetent cells. Transformants were selected after overnight growth at 37° C. on LB agar plates supplemented with ampicillin (100 mg/L) and kanamycin (100 mg/L). The resulting colonies were inoculated into 200 mL LB medium containing 100 mg/L of ampicillin and kanamycin. Protein expression was induced by 0.5 mM β-d-thiogalactopyranoside (IPTG) when the A600 reached about 0.6. The temperature was lowered to 25° C. After 12 h induction, the cells were collected, and the pellets were stored at −80° C.

About 100 mL of cell culture pellet was thawed and solubilized with ˜20 mL lysis buffer (150-1000 mM NaCl, 50 mM Tris-HCl, pH 7.5, 1 mM MgCl2, 20 mM imidazole, 0.1 units/mL DNase I, 10 μg/mL lysozyme, 1% v/v Triton X-100) and stirred with a vortex shaker for 1 hour at 22° C. The bacteria were then lysed by sonication (duty cycle 10%, output control 3, Branson Sonifier 450). The lysate was subsequently centrifuged at 6000×g at 4° C. for 20 min and the cellular debris discarded. The supernatant was mixed with 100 μL of Ni-NTA resin (Qiagen) to a 50 mL falcon tube, which was pre-equilibrated with wash buffer (1% v/v Triton X-100, 150 mM NaCl, 50 mM Tris-HCl, pH 7.5). After 1 hour, the resin was loaded into a column (Micro Bio Spin, Bio-Rad), which was pre-washed with 5 mL wash buffer (150 mM NaCl, 15 mM Tris-HCl, pH 7.5, 1% v/v Triton X-100). The protein was eluted with approximately 200 μL elution buffer (500 mM imidazole, 150-1000 mM NaCl, 15 mM Tris-HCl, pH 7.5, 1% v/v Triton X-100). Subsequently, the eluted protein was mixed with 50 μL of Strep-Tactin resin (IBA) to a 2 mL tube, which was pre-equilibrated with wash buffer (1% v/v Triton X-100, 150 mM NaCl, 15 mM Tris-HCl, pH 7.5). After 30 minutes, the resin was loaded into a column (Micro Bio Spin, Bio-Rad), which was pre-washed with 5 mL wash buffer (150 mM NaCl, 50 mM Tris-HCl, pH 7.5, 1% v/v Triton X-100). In total, 10 mL of wash buffer (150-1000 mM NaCl, 50 mM Tris, pH 7.5, 20 mM imidazole, 0.2% v/v Triton X-100) was used to wash the beads. The protein was eluted with approximately 100 μL elution buffer (2.5 mM desthiobiotin, 150-1000 mM NaCl, 50 mM Tris-HCl, pH 7.5, 0.2% v/v Triton X-100).

Proteolytic activity of artificial proteasome-nanopore (complex 3). To determine the proteolytic activity of artificial proteasome-nanopore, β-casein was incubated with purified complex 3 under a variety of incubating time, temperature, and salt concentration (FIG. 5A, FIG. 5B, FIG. 5C). Firstly, an aliquot of 0.1 mL β-casein (1 mg/mL) was incubated with complex 3 at 53° C. in buffer A (50 mM Tris, pH 7.5, 150 mM NaCl). The final β-casein/complex 3 concentration ratio was 42 (FIG. 5A). In the absence of the protease, no degradation of β-casein was observed. After 15 min of incubation at 53° C. with complex 3, almost all β-casein was digested, with about three quarters of the initially observed proteins no longer detectable on SDS-PAGE. After 30 minutes' incubation, all β-casein was digested. Then, a variety of temperature and salt concentration for degradation of β-casein were tested. As shown in FIG. 5B and FIG. 5C, the proteolytic activity increased with the temperature and decreased with increasing the salt concentration.

Electrical recordings in planar lipid bilayers. The setup consisted of two chambers separated by a 25 μm thick polytetrafluoroethylene film (Goodfellow Cambridge Limited), which contain an aperture of approximately 100 μm in diameter, which was formed by applying a high voltage spark. To form a lipid bilayer, the aperture was pre-treated with a drop of 5% hexadecane/pentane solution. After waiting about 1-5 minutes in order to allow pentane to evaporate, 500 μL of a buffered solution (150 mM NaCl, 15 mM Tris-HCl, pH 7.5) was added to each compartment. Then a drop of 1,2-diphytanoyl-sn-glycero-3-phosphocholine (DPhPC) in pentane (˜10 mg/mL) was added to each compartment. After evaporation of the pentane, a lipid monolayer formed spontaneously by pipetting the solution up and down over the aperture. Silver/silver-chloride electrodes were submerged into the solution of each compartment. Nanopores were added to the trans side. All experiments were performed at ˜23° C.³⁶.

Data recordings and analysis. Electronic signals were recorded by using an Axopatch 200B (Axon Instruments) with digitization performed with a Digidata 1440 (Axon Instruments). Clampex 10.7 software and Clampfit 10.7 software (Molecular Devices) were used for electronic signal recording and subsequent data analysis, respectively. Events were collected using the single-channel search feature in clampfit and events shorter than 0.05 ms were ignored.

Ion selectivity. The current-voltage (I-V) current traces were recorded with an automated voltage protocol that applied each potential for 0.4 s from −30 to +30 mV with 1 mV steps. Ag/AgCl electrodes were surrounded with 2.5% agarose bridges containing 2.5 M NaCl. Reversal potential was measured from extrapolation from I-V curves collected under asymmetric salt concentration condition. The experiment proceeded as follow: First an individual nanopore was reconstituted using the same buffer in both chambers (1 M NaCl, 15 mM Tris, pH 7.5, 500 μL). This allowed assessing the orientation of the nanopore and allowed balancing the electrodes. Then 500 μL solution containing 4 M NaCl, 15 mM Tris, pH 7.5 was slowly added to cis side and 500 μL of a buffered solution containing no NaCl (15 mM Tris, pH 7.5) was added to trans side (trans:cis, 2.0 M NaCl: 0.5 M NaCl).

### EXAMPLE 1: DESIGN OF AN ARTIFICIAL NANOPORE

The 20S proteasome from Thermoplasma acidophilum has a cylindrical structure made of four stacked rings composed of 14 α-and 14 β-subunits (FIG. 1E)¹². The two flanking outer α-rings allow for the association of the 20S proteasome with several regulatory complexes¹³, among which is proteasome activator PA28 (FIG. 1A) that controls the translocation of substrates into the catalytic cavity¹⁴. We designed a PA28 nanopore by replacing the disorder region in a subunit of PA28 (from 163 to P100) with the transmembrane region (VHGNAEVHASFFDIGGSVSAGF (SEQ ID NO: 6)) of anthrax protective antigen¹⁵ flanked by a short flexible linker (SSG) on each side (FIGS. 1A-1D, FIG. 2A). The 22 residues of this transmembrane (TM) region is sufficient to span the hydrophobic core of a lipid bilayer.

The amino acid sequence of a subunit of the artificial PA28-nanopore was as follows:

```
                  10 20 30 40 50 60
MATLRVHPEA QAKVDVFRED LCSKTENLLG SYFPKKISEL DAFLKEPALN EANLSNLKAP
                  70 80 90 100 110 120
      GSSVHGN AEVHASFFDI GGSVSAGFSS G
LDI CGPVNCNEK IVVLLQRLKP EIKDVTEQLN
      PVPDPVKEKEKEERKKQQEKEEKEEKKKGDEDDKGPP
                  130 140 150 160 170 180
LVTTWLQLQI PRIEDGNNFG VAVQEKVFEL MTNLHTKLEG PHTQISKYFS ERGDAVAKAA
                  190 200 210 220 230 240
KQPHVGDYRQ LVHELDEAEY QETRLMVMEI RNAYAVLYDI ILKNFEKLKK PRGETKGMIY
                  250
GSSWSHPQFE K
```

SEQ ID NOS 9-10, respectively, in order of appearance. “GSSVHGNAEVHASFFDIGGSVSAGFSSG” disclosed as SEQ ID NO: 7, and “PVPDPVKEKEKEERKKQQEKEEKEEKKKGDEDDKGPP” disclosed as SEQ ID NO: 8. The transmembrane region of protective antigen flanked by 2 short linkers (SSG) (indicated in bold) was inserted in the polypeptide sequence of PA28α, which insertion also involved deletion of the stretch of amino acids of PA28 that is indicated in italics.

In order to optimize the fusion nanopore, the length of the linker was varied by adding or removing residues on each side of the transmembrane region. One deletion mutant (Δ2) and five insertion mutants (∇2, ∇4, ∇8, ∇12, and ∇16) were prepared based on the sequence of protective antigen nanopore¹⁵ (FIG. 2A). With the exception of Δ2, all variants could insert into the lipid bilayer. However, the insertion efficiency and subsequent bilayer stability differed amongst the mutants. ∇8, ∇12, and ∇16 showed large current fluctuations, which prevented nanopore analysis, suggesting the linker introduces a large conformational flexibility to the nanopore. ∇4 showed low-noise conductance with occasional full current blocks at positive applied potentials. However, the nanopores showed a heterogeneous unitary conductance and often closed at negative applied potentials (FIG. 2B). Among all the constructs, ∇2, which was efficiently expressed and purified, produced the most uniform pores in lipid bilayers (mean unitary conductance of 1.17±0.14 nS at −35 mV, 1 M NaCl, 15 mM Tris, pH 7.5, n=59, FIG. 2C).

Remarkably, ∇2 inserted as efficiently and as uniformly as other nanopores found in nature (e.g. alpha hemolysin16). The individual peptides corresponding to the TM region of anthrax protective antigen could not form nanopores, indicating that a soluble scaffold is required to stabilize the nanopore in lipid bilayers.

Molecular dynamics (MD) simulations were performed on the ∇2 PA-nanopore (hereafter PA-nanopore) to better understand the electrostatic and hydrophobic Interactions between the nanopore and the lipid bilayer. As shown in FIG. 2D, two rings of hydrophobic residues anchor the TM region to the hydrophobic edges of the bilayer, while alternated residues with aliphatic side-chains interface the core of the bilayer. The lumen of the pore is kept hydrated by hydrophilic residues. As expected, the hydrophilic side-chain of the linker residues are interacting with the charged head groups of membrane lipids.

### EXAMPLE 2: ELECTRICAL AND FUNCTIONAL PROPERTIES OF THE OPTIMIZED ARTIFICIAL PORE

Similar to other β-barrel nanopores such as αHL¹⁸ and aerolysin¹⁹ nanopore, the artificial PA-nanopore showed an asymmetric current-voltage (I-V) relationship (FIG. 3C), which allowed identifying the orientation of the pore in the lipid bilayer. Ion-selectivity measurements using asymmetric NaCl concentrations (0.5 M/cis and 2 M/trans) revealed a cation selective nanopore (PK⁺PCl⁻=1.76±0.20, FIG. 3D). Here and throughout the manuscript, errors indicate the standard deviations obtained from three experiments. The correct folding of the PA-nanopore was characterized using cyclodextrins (CDs), circular molecules that binds to β-barrel nanopores²⁰. α-CD, β-CD and γ-CD were added to the cis side of the artificial nanopore and the magnitude of the ionic current associated with a blockade (I_{B}) was measured. To characterize the blockade, we used the percentage of excluded current (I_{res %}), defined as [(I_{O}−I_{B})/I_{O}]×100, where I_{O} represents the open pore current. α-CD most likely translocated across the nanopore too quickly, as no current blockades were observed. By contrast, β-CD and γ-CD showed characteristic blockades (FIG. 3E and FIG. 3F). Finally, the ability of the nanopore to identify peptides was tested using angiotensin I and dynorphin A. We found that the two peptides induced blockades which could be easily distinguished using several parameters, including the residual current and the duration of the current blockades (FIG. 3G and FIG. 3H).

### EXAMPLE 3: DESIGN OF AN ARTIFICIAL TRANSMEMBRANE PROTEASOME

In cells, PA28 docks onto the 20S proteasome and controls the translocation of substrates into the catalytic cavity²¹. We found, however, that when the proteasome was added to the cis side of individual PA28-nanopores in 1 M NaCl solutions, no clear interaction was observed. Most likely, the high ionic strength used do not allow such interaction²². The crystal structure of the Thermoplasma acidophilum proteasome in complex with PA26 from Trypanosoma brucei²³, a homolog of PA28, shows that the carboxy-terminal tails of PA26 slide into a pocket on the 20S proteasome, near the amino-terminus of the α subunit (FIG. 4A). Hence, we fused the C-terminal of PA28 (S231) with L21 of the proteasome α subunit. In the designed protein complex the first 20 residues of the α subunit are removed, leaving the proteasome gate open towards the PA28 nanopore. The proper assembly of the proteasome requires co-assembly of the α and β subunits. Thus, PA28 fused to proteasome Δ20-α subunit (PA28-αΔ20 nanopore) containing an N-terminal His-tag was cloned into pET-28a vector, carrying a gene for kanamycin resistance. The proteasomal αΔ12, containing a C-terminal Strep-tag, and β subunit were both cloned into a pETDuet-1 vector, carrying a gene for kanamycin resistance (FIG. 4B). In αΔ12 the first 12 residues of the α subunit were removed allowing fast degradation of unfolded substrates without the need for a proteasome activator²⁴. The co-assembled proteasome-nanopore was then purified in two steps by affinity chromatography using 1 M NaCl, 50 mM Tris, pH 7.5 solutions (FIG. 4B). SDS-PAGE and native PAGE confirmed the successful assembly of the multi-protein complex (FIG. 4C). Activity assays revealed that the proteasome nanopore was active, with the proteolytic activity increasing with the temperature and decreasing with the salt concentration (FIG. 5). The transmembrane proteasome inserted efficiently in lipid bilayers and showed low-noise current recordings, albeit some extent of fast gating at positive potentials was observed (FIG. 4D). The I-V curve of the proteasome-nanopore in 1 M NaCl solutions was similar to that of PA-nanopore (data not shown), suggesting that the transmembrane region was unchanged and the gate of the α-subunit was open. These results suggest that co-expression and two-step purification procedure can be used for the effective isolation of stable subcomplex 3 (PAαΔ20-ββ-αΔ12 nanopore) formed in E. coli. in solutions containing 1 M NaCl.

### EXAMPLE 4: REAL-TIME PROTEIN PROCESSING

The activity of the transmembrane proteasome was tested using substrates containing a C-terminal ssrA tag, which mediates the interaction with VAT (Valosin-containing protein-like ATPase of Thermoplasma acidophilum)²⁵, an unfoldase that threads substrate proteins through the proteasome chamber. The first substrate, named S1, was 123 amino acid long and was designed to be unstructured and to contain four stretches of 15 serine residues (SEQ ID NO: 41) flanked by a group of 10 arginines (SEQ ID NO: 42) and three hydrophobic residues. The second substrate was S2, a longer polypeptide of 210 amino acids. The third substrate was green fluorescent protein (GFP)²⁵ carrying 10 arginines (SEQ ID NO: 42) and an ssrA tag (AANDENYALAA (SEQ ID NO: 11)) at the C-terminus.

S1:
1 11 21 31 41 51 61
MGHHHHHHSS RRRPRRRPRR SSSSSSSSSS SSSSSFGYGW SSSSSSSSSS SSSSSRRRRR RRRRPSSSSS
71 81 91 101 111 121
SSSSSSSSSS FGYGWSSSSS SSSSSSSSSS RRRRRRRRRR SSAANDENYA LAA
S2:
1 11 21 31 41 51 61
MGHHHHHHSS RRRRRRVPLP IPVPLPIPVP LPIPRRRRRS SSSSSSSSSS SSSSSSSSSS SSSSSSSSSS
71 81 91 101 111 121 131
SSSSSSSSSS SSSSSSSSSR RRRRPVPLPI PVPLPIPVPL PIPRRRRRSS SSSSSSSSSS SSSSSSSSSS
141 151 161 171 181 191 201
SSSSSSSSSS SSSSSSSSSS SSSSSSSSEE EEEPVPLPIP VPLPIPVPLP IPEEEEESSA ANDENYALAA
S3:
1 11 21 31 41 51 61
MGHHHHHHSS SKGEELFTGV VPILVELDGD VNGHKFSVSG EGEGDATYGK LTLKFICTTG KLPVPWPTLV
71 81 91 101 111 121 131
TTLTYGVQCF SRYPDHMKRH DFFKSAMPEG YVQERTISFK DDGNYKTRAE VKPEGDTLVN RIELKGIDFK
141 151 161 171 181 191 201
EDGNILGHKL EYNYNSHNVY ITADKQKNGI KANFKIPHNI EDGSVQLADH YQQNTPIGDG PVLLFDNHYL
211 221 231 241 251
STQSALSKDP NEKRDHMVLL EFVTAAGITH GMDELYKSSA ANDENYALAA

SEQ ID NOS 12-14, respectively, in order of appearance.

Initial tests were performed using a transmembrane proteasome, in which the proteolytic activity was removed by substituting the amino-terminal threonine 1 in the active site with alanine²⁶. Reactions were performed in 1 M NaCl, 15 mM Tris-HCl, pH 7.5, 20 mM MgCl₂ solutions. The addition of 20.0 μM of S1 to the cis compartment of an inactive proteasome-nanopore induced both short (average dwell time is 0.62±0.11 ms) and second-long current blockades (FIG. 6A). Most likely, the short events represent the substrate either translocating across the nanopore, and the long events the substrate remaining blocked within the proteasome chamber. Both blockades showed a residual current close to zero (Iᵣₑₛ %=11.56±0.13), suggesting that during translocation the unstructured substrates occluded most of the nanopore. When VAT (20.0 μM) was added in solution in the presence of 2.0 mM ATP, the second-long blockades were no longer observed (FIG. 6B). Furthermore, more ionic current was observed during the VAT-assisted translocation events compared to un-assisted translocation events (Iᵣₑₛ %=83.81±0.11), suggesting that the substrate was stretched while VAT unfolded the substrate. Several recurring current signatures were observed during translocation (average dwell time is 5.8±3.9 ms), suggesting that the different features of the substrate are reflected in the ionic signal (FIG. 6B).

When a GFP was used instead of S1, the current blockades became longer (average dwell time is 22.1±20.2 ms) and the current signature was strikingly different compared with S1 (FIG. 6B, FIG. 6C), indicating that the two substrates can be differentiated based on their ionic current signal. When the ATP concentration was increased to 6.0 mM, the average dwell time of GFP blockades decreased 10-fold to 2.4±1.7 ms (data not shown). Hence, VAT is capable of feeding the polypeptide through the nanopore at a speed that can be tuned by the concentration of ATP.

When the active proteasome was used in the presence of S1 but in the absence of VAT and ATP, uniform and short blockades were observed (FIG. 6D). Their average dwell time (0.51±0.03 ms) was shorter than that observed for the analogous events recorded with the inactive proteasome, suggesting that the proteasome processed at least in part the substrate during translocation. When a longer unfolded substrate was tested (S2), the average dwell time of the observed events was longer (2.26±0.26 ms) and deeper residual currents were observed compared to S1, indicating that larger polypeptide fragments are formed. Mixtures of S1 and S2 could be readily distinguished by ionic current blockades. interestingly, when S1 was tested with VAT (20.0 μM) and ATP (2.0 mM), more spaced and shorter blockades were observed (FIG. 6E), suggesting that the reduced speed of polypeptide threading across the proteasomal chamber allowed more efficient degradation of the polypeptide into small peptides that are quickly transported across the nanopore. Accordingly, when GFP was tested under the same conditions no blockades were observed, suggesting that the slower unfolding of GFP compared to the unstructured S1 allowed for a yet more efficient proteolysis of the substrate into yet smaller peptides. These peptides are transported across the nanopore too quickly to be observed²⁷.

### EXAMPLE 5: PA26-Artificial Nanopore

This example describes the design and characterization of an artificial nanopore comprising the ring-forming multimeric proteasome activator protein PA26, which is a homolog of PA28.

The transmembrane sequence (bold) of anthrax protective antigen (PDB ID: 3J9C) was fused in the middle of a subunit of PA26 (PDB ID: 1YA7), from which the 12-amino acid sequence shown in italics was deleted, via 2 linkers (GSSSE-SNSSG (“GSSSE” disclosed as SEQ ID NO: 15, “SNSSG” disclosed as SEQ ID NO: 16).

The complete sequence of an N-terminally Strep-tagged subunit of the artificial PA26-nanopore is as follows:

```
                  10 20 30 40 50 60 70
MGWSHPQFEK SSGPFKRALL IQNLRDSYTE TSSFAVIEEW AAGTLQEIEG IAKAAAEAHG VIRNSTYGRA
                  70 80 90 100 110 120
QAEKSPEQLL GVLQRYQDLC HNVYCQAETI RTVIAIRIPE HKEEDNLGVA VQHAVLKIID ELEIKTLGSG
                  130 140 150 160 170 180
                                                                        GSSSEVH GNAEVHASFF DIGGSVSAGF SNSSG
EKGGSGGAPT PIGMYALREY LSARSTVEDK LLG SQSPS
                                                                        SVDAESGKTKGG
                220 230 240 250 260
LLLELRQIDA DFMLKVELAT THLSTMVRAV INAYLLNWKK LIQPRTGSDH MVS
```

SEQ ID NOS 19-20, respectively, in order of appearance. “GSSSEVHGNAEVHASFFDIGGSVSAGFSNS SG” disclosed as SEQ ID NO: 17, and “SVDAESGKTKGG” disclosed as SEQ ID NO: 18. FIG. 8A, FIG. 8B, FIG. 8C, FIG. 8D shows the structure of the resulting artificial PA26-nanopore, and typical current trace demonstrating insertion of an individual pore.

### EXAMPLE 6: ATPASE-ARTIFICIAL NANOPORE

This example describes the design and characterization of an artificial nanopore comprising the ring-forming multimeric Aquifex aeolicus ATPase (PDB ID: 3M0E), as an example of a protein capable of transporting a polynucleotide.

The transmembrane sequence (bold) of anthrax protective antigen (PDB ID: 3J9C) was inserted in the middle of a subunit of the ATPase, from which the amino acid sequence indicated in italics was deleted (insertional replacement). The inserted TM sequence was flanked on both sides with a linker (SSSSS (SEQ ID NO: 21)) as indicated in bold. The complete sequence of an N-terminally Strep-tagged a subunit of the artificial ATPase-nanopore is as follows:

```
                10 20 30 40 50 60
MGWSHPQFEK SSGRKENELL RREKDLKEEE YVFESPKMKE ILEKIKKISC AECPVLITGE
                70 80 90 100 110 120
                                                                                                SSSSSV HGNAEVHASF
SGVGKEVVAR LIHKLSDRSK EPFVALNVAS IPRDIFEAEL FGYE
                                                                                                KGAFTGAVS
              130 140 150 160 170 180
FDIGGSVSAG FSSSSS
                                  SKEG FFELADGGTL FLDAIGELSL EAQAKLLRVI ESGKFYRLGG
              190 200 210 220 230 240
RKEIEVNVRI LAATNRNIKE LVKEGKFRED LYYRLGVIEI EIPPLRERKE DIIPLANHFL
              250 260 270 280 290 300
KKFSRKYAKE VEGFTKSAQE LLLSYPWYGN VRELKNVIER AVLFSEGKFI DRGELSCLVN
```

SEQ ID NOS 24-25, respectively, in order of appearance. “SSSSSVHGNAEVHASFFDIGGSVSAGFSSSSS” disclosed as SEQ ID NO: 22, and “KGAFTGAVS” disclosed as SEQ ID NO: 23. FIG. 9A, FIG. 9B, FIG. 9C, FIG. 9D shows the structure of the assembled subunits to provide an artificial ATPase transmembrane nanopore. Rewardingly, the artificial ATPase nanopore could be efficiently expressed and reconstituted into lipid bilayers to form nanopores. Addition of ATP to the solution increased the noise of the baseline nanopore, indicating that the protein was active. Herewith, another example of an artificial nanopore is provided that is based on the fusion of a beta barrel to a toroidal protein.

### EXAMPLE 7: CLPP-ARTIFICIAL NANOPORE

This example describes the design of an artificial nanopore for single-molecule protein analysis. It is based on an artificial PA28-nanopore as described in Example 1, fused at its N-terminus to a subunit of ClpP. ClpP (PDB ID: 1TYF) is the caseinolytic Clp protease (ClpP) from E. coli. Wang et al. (1997) Cell 91:447-456) determined the structure of ClpP at 2.3 Å resolution. The active protease resembles a hollow, solid-walled cylinder composed of two 7-fold symmetric rings stacked back-to-back. Its 14 proteolytic active sites are located within a central, roughly spherical chamber approximately 51 Å in diameter. Access to the proteolytic chamber is controlled by two axial pores, each having a minimum diameter of approximately 10 Å.

The complete sequence of a C-terminally Strep-tagged subunit of the artificial ClpP-nanopore is as follows:

```
                10 20 30 40 50 60
MGSYSGERDN FAPHMALVPM VIEQTSRGER SFDIYSRLLK ERVIFLTGQV EDHMANLIVA
                70 80 90 100 110 120
QMLFLEAENP EKDIYLYINS PGGVITAGMS IYDTMQFIKP DVSTICMGQA ASMGAFLLTA
              130 140 150 160 170 180
GAKGKRFCLP NSRVMIRQPL GGYQGQATDI EIHAREILKV KGRMNELMAL HTGQSLEQIE
              190 200 210 220 230 240
RDTERDRFLS APEAVEYGLV DSILTHRNAT LRVHPEAQAK VDVFREDLCS KTENLLGSYF
              250 260 270 280 290 380
PKKISELDAF LEKPALNEAN LSNLKAPLDI GSSSEVHGNA EVHASFFDIG GSVSAGFSNS
              310 320 330 340 350 360
SGCGPVNCNE KIVVLLQRLK PEIKDVTEQL NLVTTWLQLQ IPRIEDGNNF GVAVQEKVPE
              370 380 390 400 410 420
LMTNLHTKLE GFHTQISKYF SERGDAVAKA AKQPHVGDYR QLVRELDEAE YQEIRLMVME
              430 440 450 480
IPNAYAVLYD IILKNFEKLK KPRGETKGMI YGSSWSHPQF EK
```

Figure discloses SEQ ID NO: 26. Residues 1-208 (italics) represent the primary sequence of ClpP from E. coli; residues 209-462 is the PA-nanopore including the C-terminal Strep-tag peptide WSHPQFEK (SEQ ID NO: 37); underlined residues 271-273 and 300-302 are linkers; and residues 274-299 (bold) represent the TM region.

FIG. 10A, FIG. 10B, FIG. 10C, FIG. 10D, FIG. 10E, and FIG. 10F depict the schematic design of the artificial ClpP-nanopore.

SDS-PAGE analyses of the purified ClpP-nanopore the presence of two unique bands corresponding well the molecular weights of active ClpP-Papore, active ClpP, inactive ClpP-Papore, and inactive ClpPPAαΔ20 (data not shown).

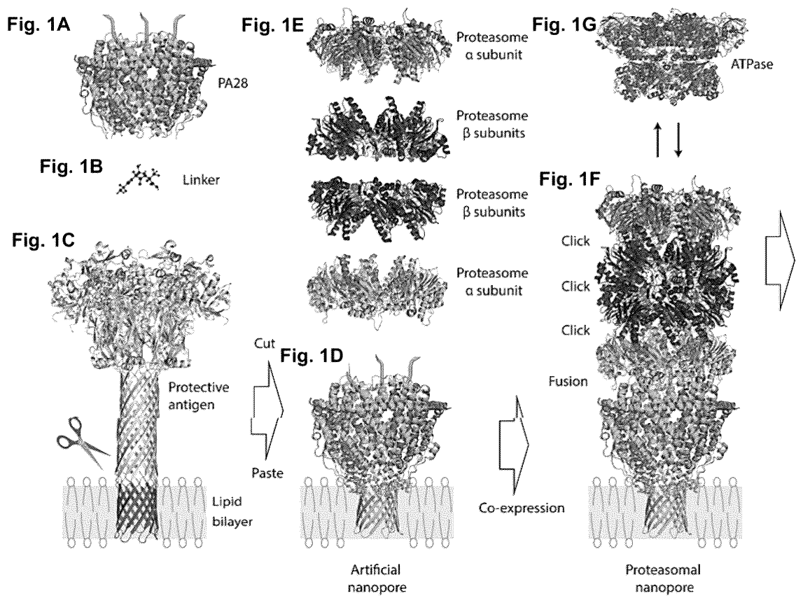

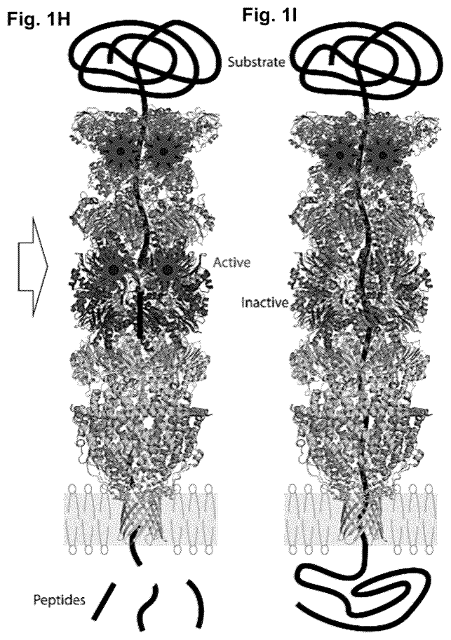

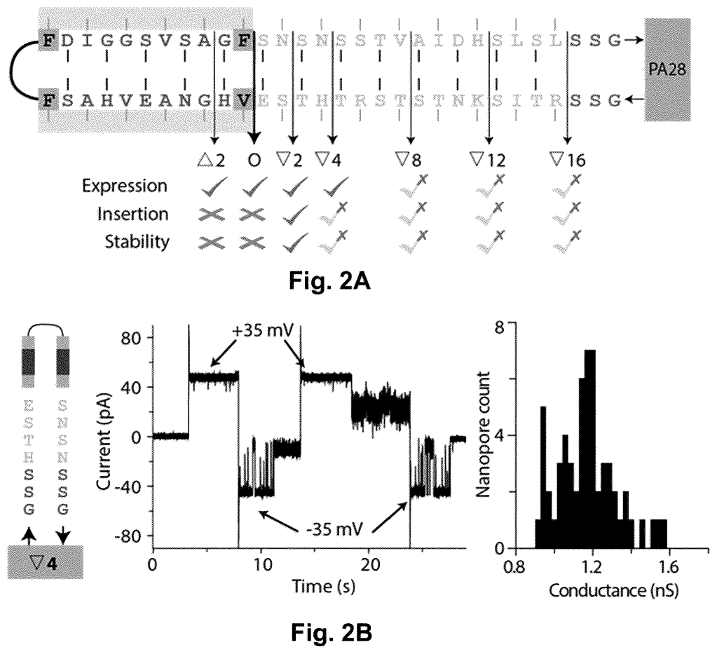

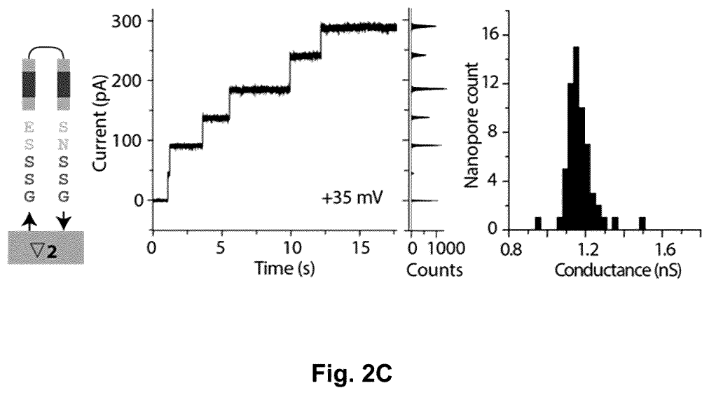

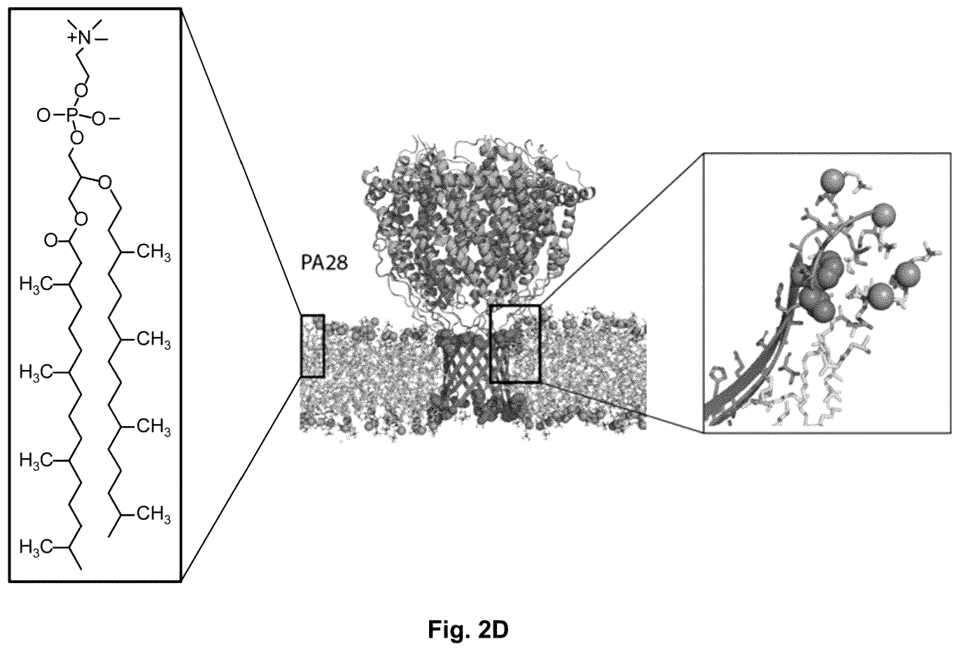

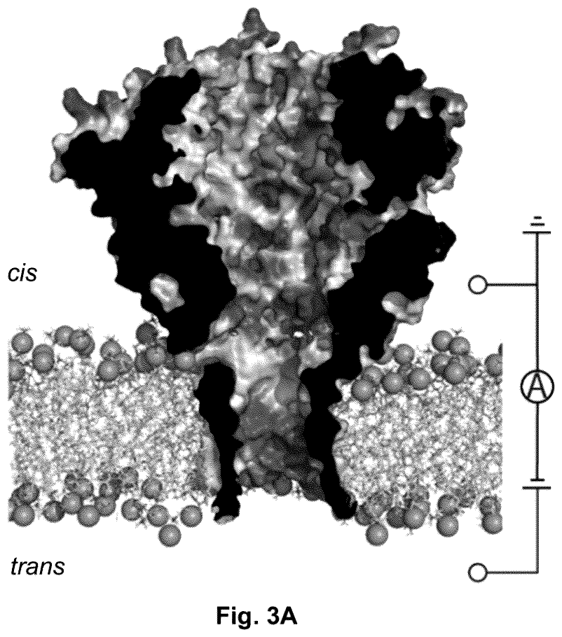

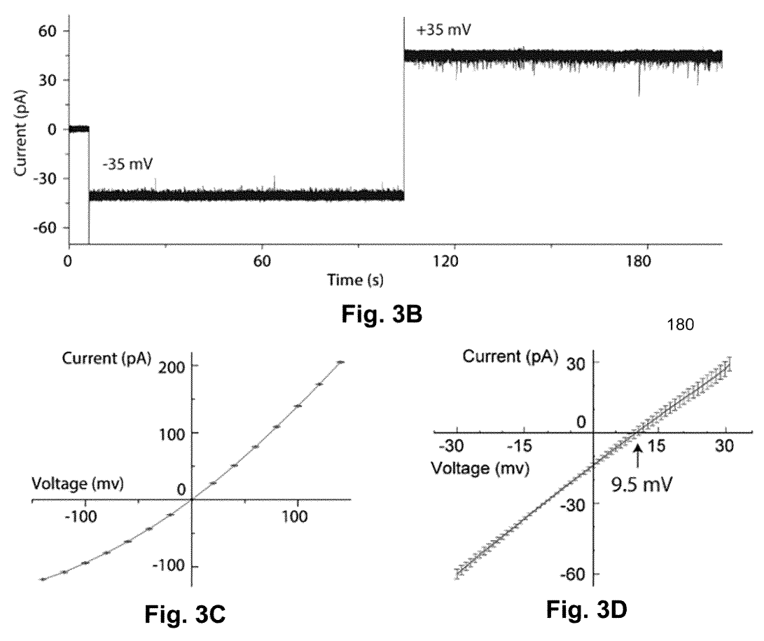

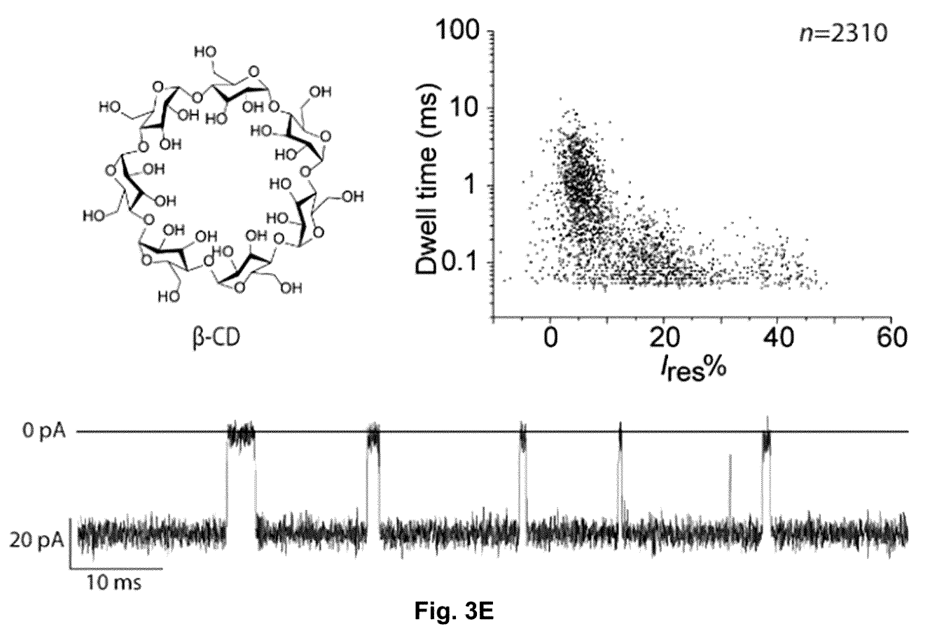

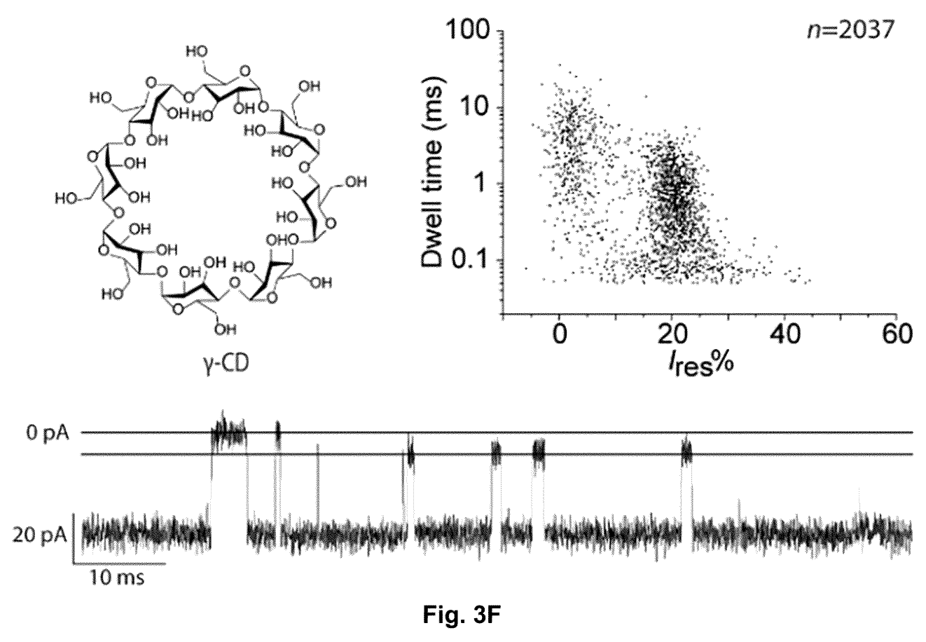

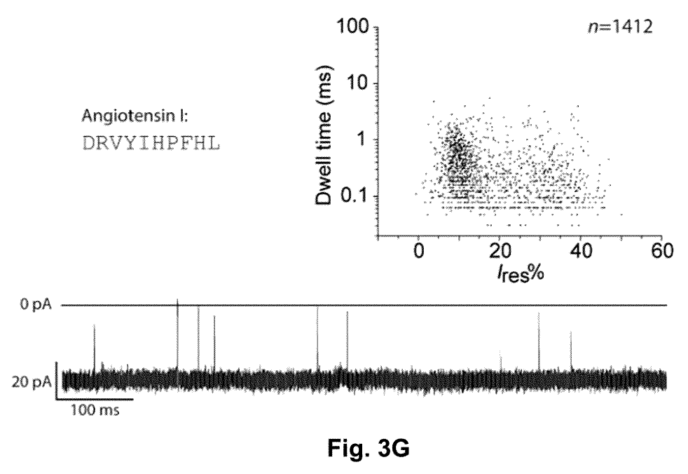

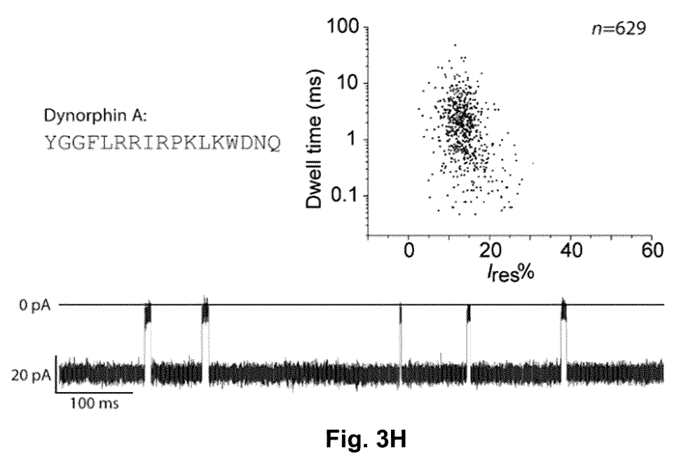

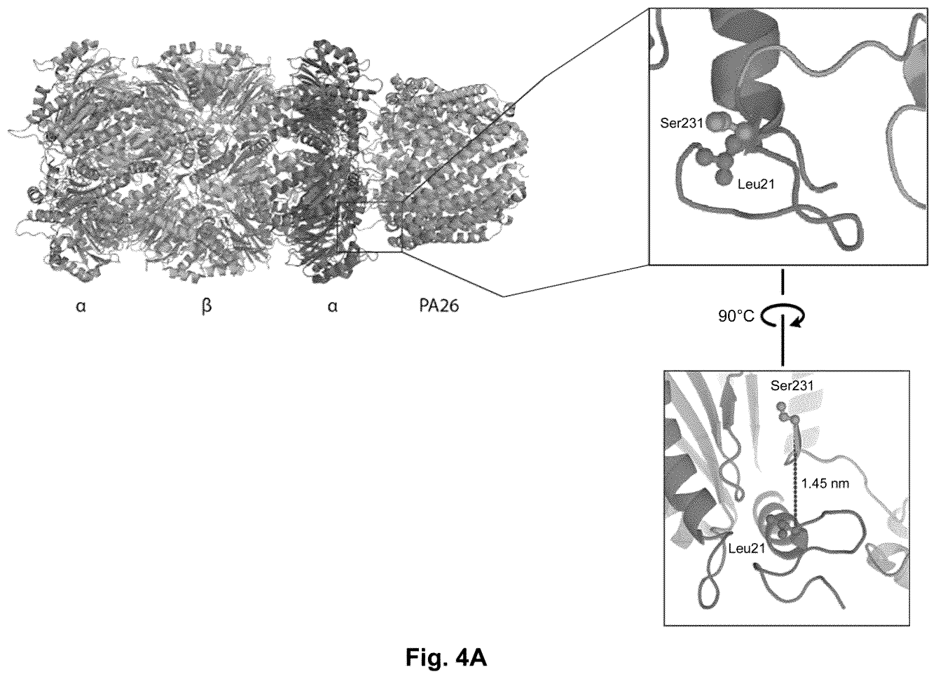

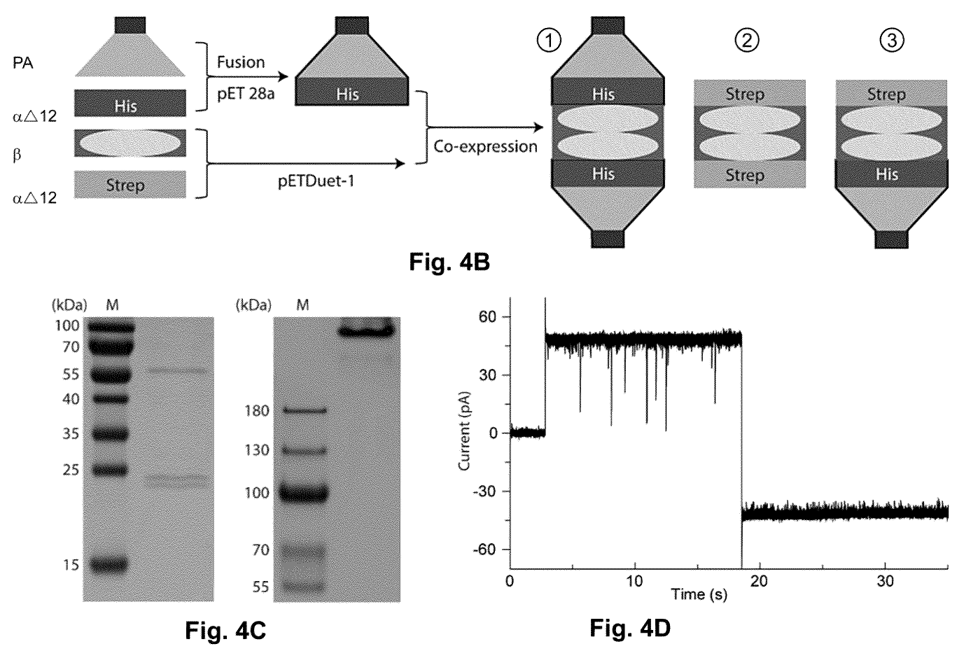

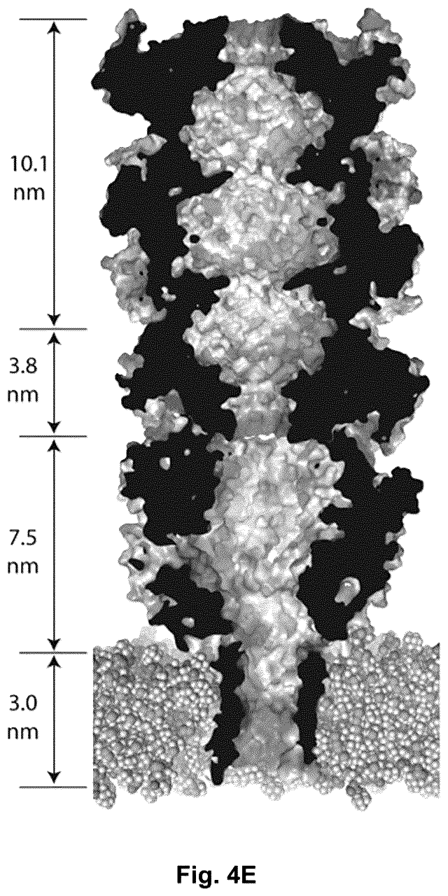

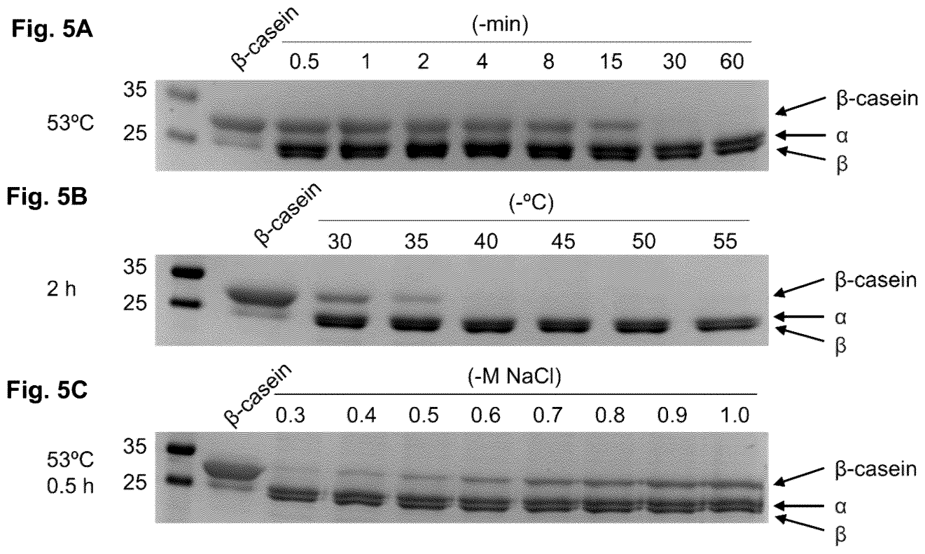

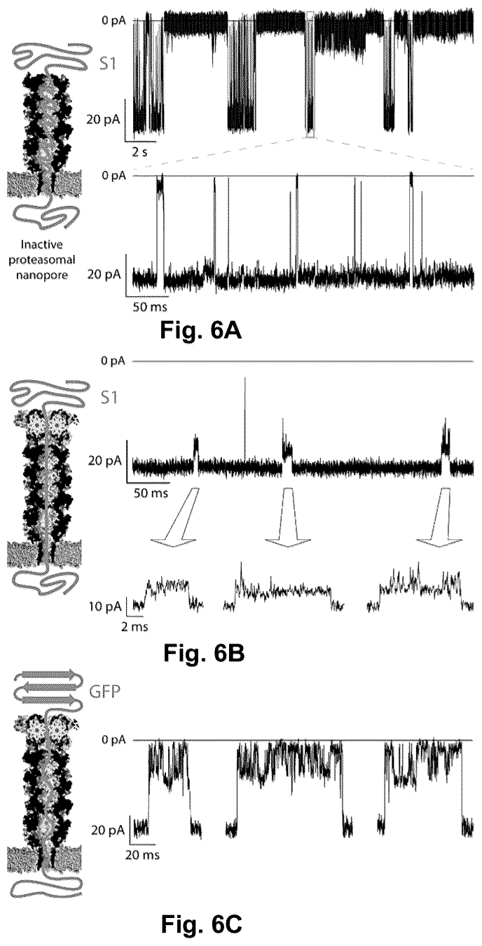

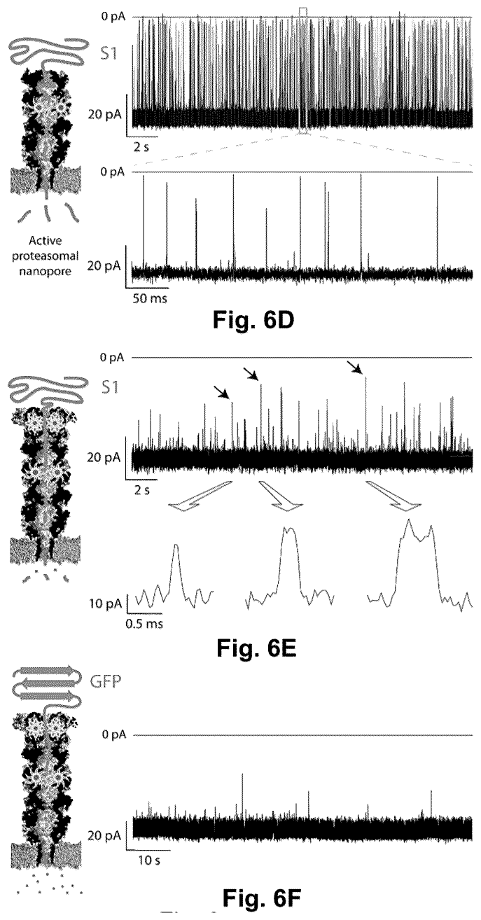

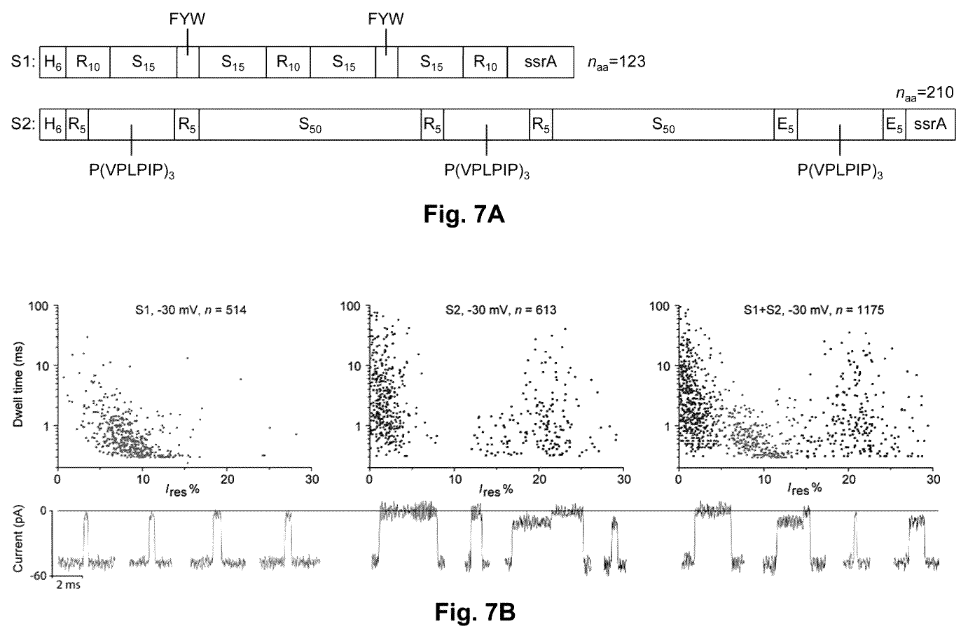

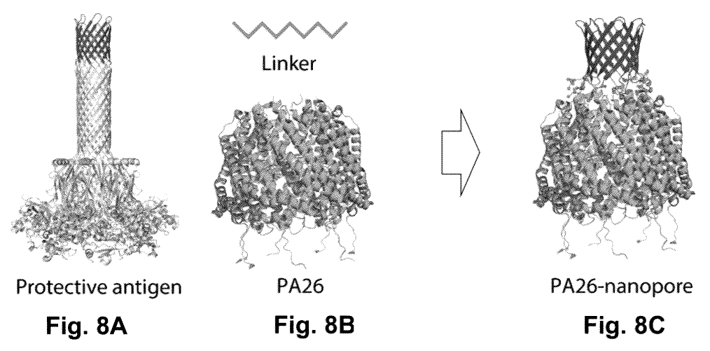

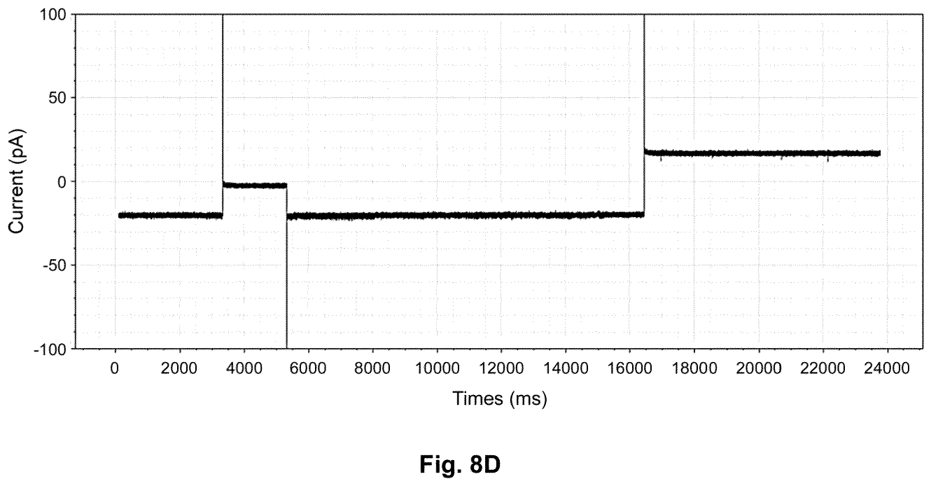

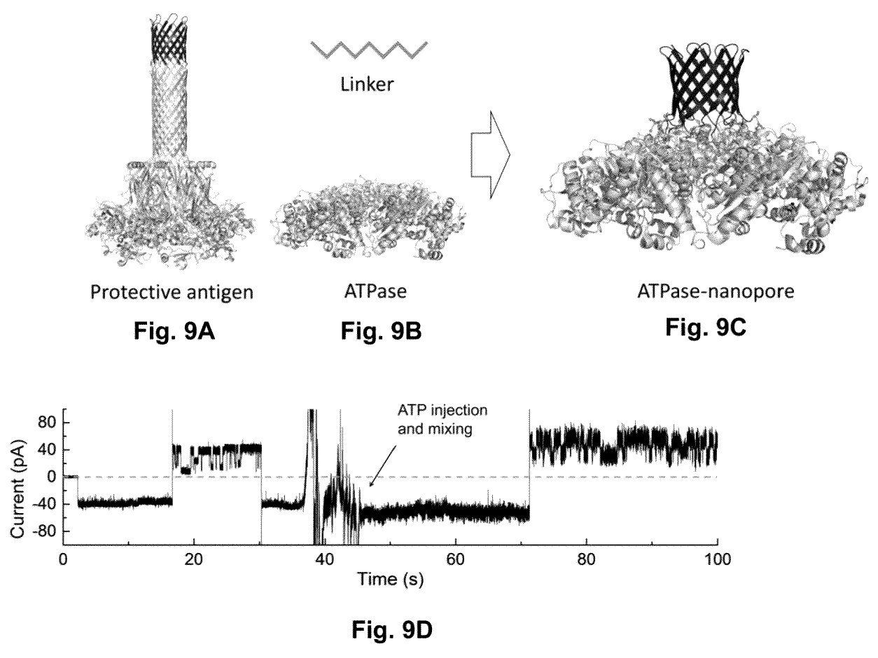

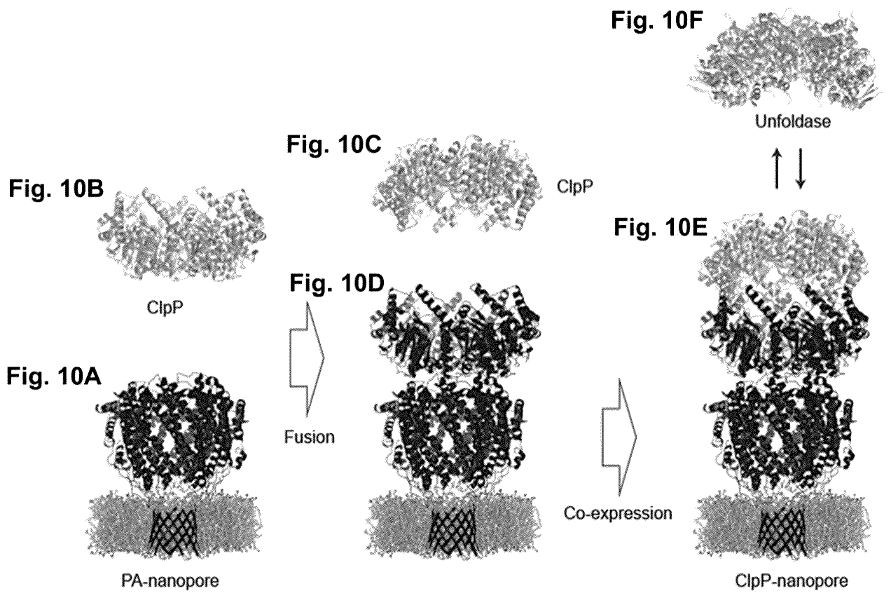

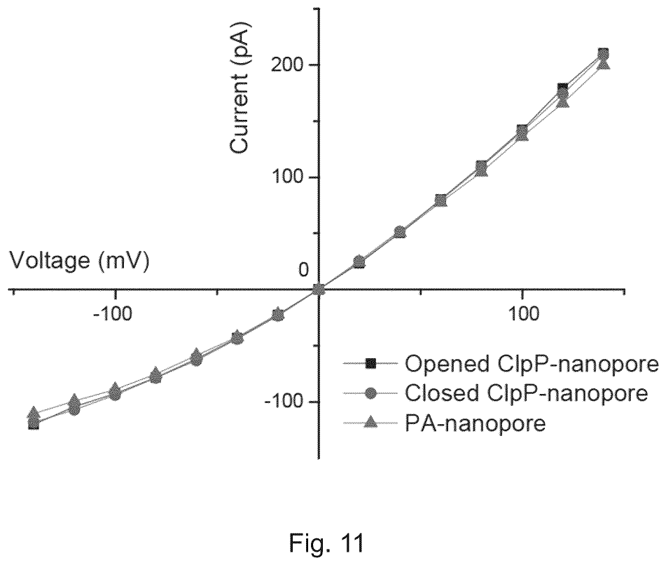

FIG. 11 shows current-voltage (I-V) characteristics of three different nanopores. The artificial opened and closed ClpP-nanopore did not alter the conductance of the nanopore. The current signals were recorded in 0.5 M KCl, 20 mM HEPES, pH 7.5, filtered at 2 kHz, and sampled at 10 kHz.

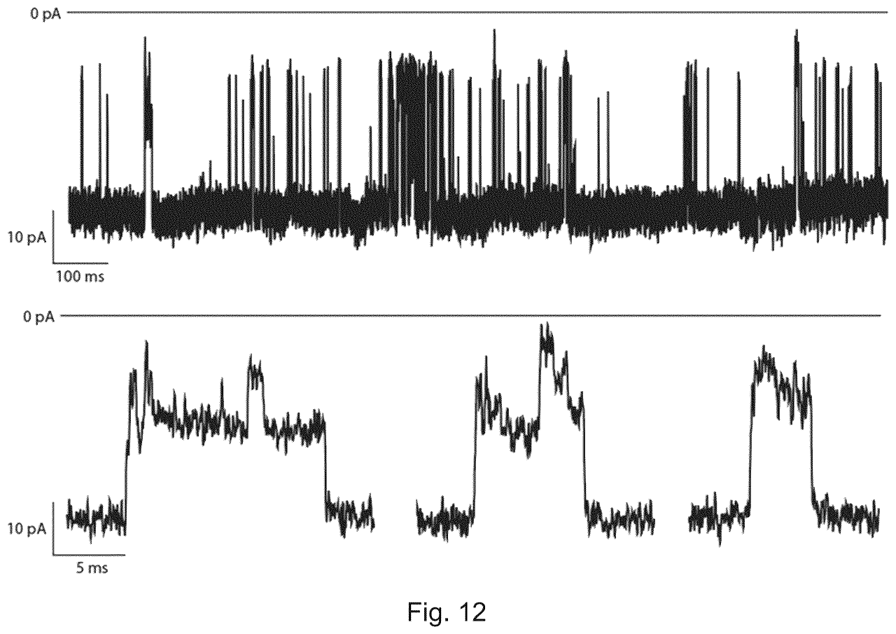

FIG. 12 shows the controlled translocation of a protein (GFP) through the ClpP-nanopore. ClpX-assisted transport of GFP across opened ClpP-nanopore in the presence of ATP. The ClpP-nanopore, ClpX and GFP were added to the cis side.

### References

1. Manrao, E. A., Derrington, I. M., Laszlo, A. H., Langford, K. W., Hopper, M. K., Gillgren, N., Pavlenok, M., Niederweis, M. and Gundlach, J. H. Reading DNA at single-nucleotide resolution with a mutant MspA nanopore and phi29 DNA polymerase. Nat. Biotechnol. 30, 349-353 (2012).
2. Noakes, M. T., Brinkerhoff, H., Laszlo, A. H., Derrington, I. M., Langford, K. W., Mount, J. W., Bowman, J. L., Baker, K. S., Doering, K. M., Tickman, B. I. and Gundlach, J. H. Increasing the accuracy of nanopore DNA sequencing using a time-varying cross membrane voltage. Nat. Biotechnol. 37, 651-656 (2019).
3. Cressiot, B., Oukhaled, A., Patriarche, G., Pastoriza-Gallego, M., Betton, J. M., Auvray, L., Muthukumar, M., Bacri, L. and Pelta, J. Protein transport through a narrow solid-state nanopore at high voltage: Experiments and theory. ACS Nano 6, 6236-6243 (2012).
4. Burns, J. R., Göpfrich, K., Wood, J. W., Thacker, V. V., Stulz, E., Keyser, U. F. and Howorka, S. Lipid-bilayer-spanning DNA nanopores with a bifunctional porphyrin anchor. Angew. Chemie-Int. Ed. 52, 12069-12072 (2013).
5. Spruijt, E., Tusk, S. E. & Bayley, H. DNA scaffolds support stable and uniform peptide nanopores. Nat. Nanotechnol. 13, 739-745 (2018).
6. Wei, B., Dai, M. & Yin, P. Complex shapes self-assembled from single-stranded DNA tiles. Nature 485, 623-626 (2012).
7. Mitchell, J. S., Glowacki, J., Grandchamp, A. E., Manning, R. S. & Maddocks, J. H. Sequence-dependent persistence lengths of DNA. J. Chem. Theory Comput. 13, 1539-1555 (2017).
8. Manning, G. S. The persistence length of DNA is reached from the persistence length of its null isomer through an internal electrostatic stretching force. Biophys. J. 91, 3607-3616 (2006).
9. Yusupov, M. M., Yusupova, G. Z., Baucom, A., Lieberman, K., Earnest, T. N., Cate, J. H. D., & Noller, H. F. Crystal structure of the ribosome at 5.5 Å resolution. Science 292, 883-896 (2001).
10. Mishra, R., Upadhyay, A., Prajapati, V. K. & Mishra, A. Proteasome-mediated proteostasis: Novel medicinal and pharmacological strategies for diseases. Med. Res. Rev. 38, 1916-1973 (2018).
11. Becker, S. H., & Darwin, K. H. Bacterial proteasomes: mechanistic and functional insights. Microbiol. Mol. Biol. Rev. 81, 1-20 (2017).
12. Löwe, J. et al. Crystal structure of the 20S proteasome from the archaeon T. acidophilum at 3.4 Å resolution. Science. 268, 533-539 (1995).
13. Förster, A. & Hill, C. P. Proteasome Activators. Protein Degrad. 2, 89-110 (2007).
14. Huber, E. M. & Groll, M. The Mammalian Proteasome Activator PA28 Forms an Asymmetric α4β3 Complex. Structure 25, 1473-1480 (2017).
15. Jiang, J., Pentelute, B. L., Collier, R. J. & Hong Zhou, Z. Atomic structure of anthrax protective antigen pore elucidates toxin translocation. Nature 521, 545-549 (2015).
16. Maglia, G., Restrepo, M. R., Mikhailova, E. & Bayley, H. Enhanced translocation of single DNA molecules through a-hemolysin nanopores by manipulation of internal charge. Proc. Natl. Acad. Sci. U. S. A. 105, 19720-19725 (2008).
17. Chen, B., Sysoeva, T. A., Chowdhury, S., Guo, L., De Carlo, S., Hanson, J. A., Yang, H. and Nixon, B.T., 2010. Engagement of arginine finger to ATP triggers large conformational changes in NtrC1 AAA+ ATPase for remodeling bacterial RNA polymerase. Structure 18, 1420-1430 (2010).
18. Stoddart, D., Ayub, M., Hofler, L., Raychaudhuri, P., Klingelhoefer, J. W., Maglia, G., Heron, A. and Bayley, H. Functional truncated membrane pores. Proc. Natl. Acad. Sci. U. S. A. 111, 2425-2430 (2014).
19. Piguet, F., Ouldali, H., Pastoriza-Gallego, M., Manivet, P., Pelta, J., & Oukhaled, A. Identification of single amino acid differences in uniformly charged homopolymeric peptides with aerolysin nanopore. Nat. Commun. 9, 966 (2018).
20. Gu, L. Q., Braha, O., Conlan, S., Cheley, S. & Bayley, H. Stochastic sensing of organic analytes by a pore-forming protein containing a molecular adapter. Nature 398, 686-690 (1999).
21. Sugiyama, M., Sahashi, H., Kurimoto, E., Takata, S. I., Yagi, H., Kanai, K., Sakata, E., Minami, Y., Tanaka, K. and Kato, K. Spatial arrangement and functional role of α subunits of proteasome activator PA28 in hetero-oligomeric form. Biochem. Biophys. Res. Commun. 432, 141-145 (2013).
22. Kuehn, L. & Dahlmann, B. Proteasome activator PA28 and its interaction with 20 S proteasomes. Arch. Biochem. Biophys. 329, 87-96 (1996).
23. Förster, A., Masters, E. I., Whitby, F. G., Robinson, H. & Hill, C. P. The 1.9 Å structure of a proteasome-11S activator complex and implications for proteasome-PAN/PA700 interactions. Mol. Cell 18, 589-599 (2005).
24. Benaroudj, N., Zwickl, P., Seemüller, E., Baumeister, W. & Goldberg, A. L. ATP hydrolysis by the proteasome regulatory complex PAN serves multiple functions in protein degradation. Mol. Cell 11, 69-78 (2003).
25. Huang, R., Ripstein, Z. A., Augustyniak, R., Lazniewski, M., Ginalski, K., Kay, L. E. and Rubinstein, J. L. Unfolding the mechanism of the AAA+ unfoldase VAT by a combined cryo-EM, solution NMR study. Proc. Natl. Acad. Sci. U. S. A. 113, E4090-W4199 (2016).
26. Seemuller, E., Lupas, A., Stock, D., Lowe, J., Huber, R. and Baumeister, W. Proteasome from Thermoplasma acidophilum: A Threonine Protease. Science 268, 579-582 (2016).
27. Kim, Y. I., Burton, R. E., Burton, B. M., Sauer, R. T. & Baker, T. A. Dynamics of substrate denaturation and translocation by the ClpXP degradation machine. Mol. Cell 5, 639-648 (2000).
28. Akopian, T. N., Kisselev, A. F. & Goldberg, A. L. Processive degradation of proteins and other catalytic properties of the proteasome from Thermoplasma acidophilum. J. Biol. Chem. 272, 1791-1798 (1997).
29. Huang, G., Voet, A. & Maglia, G. FraC nanopores with adjustable diameter identify the mass of opposite-charge peptides with 44 dalton resolution. Nat. Commun. 10, 1-10 (2019).
30. Kisselev, A. F., Songyang, Z. & Goldberg, A. L. Why does threonine, and not serine, function as the active site nucleophile in proteasomes? J. Biol. Chem. 275, 14831-14837 (2000).
31. Huber, E. M., Heinemeyer, W., Li, X., Arendt, C. S., Hochstrasser, M. and Groll, M. A unified mechanism for proteolysis and autocatalytic activation in the 20S proteasome. Nat. Commun. 7, 1-10 (2016).
32. Ripstein, Z. A., Huang, R., Augustyniak, R., Kay, L. E. & Rubinstein, J. L. Structure of a AAA+ unfoldase in the process of unfolding substrate. Elife 6, 1-14 (2017).
33. Miles, G., Cheley, S., Braha, O. & Bayley, H. The staphylococcal leukocidin bicomponent toxin forms large ionic channels. Biochemistry 40, 8514-8522 (2001).
34. Horton, R. M., Hunt, H. D., Ho, S. N., Pullen, J. K. & Pease, L. R. Engineering hybrid genes without the use of restriction enzymes: gene splicing by overlap extension. Gene 77, 61-68 (1989).
35. Liu, H. & Naismith, J. H. An efficient one-step site-directed deletion, insertion, single and multiple-site plasmid mutagenesis protocol. BMC Biotechnol. 8, 91 (2008).
36. Maglia, G., Heron, A. J., Stoddart, D., Japrung, D. & Bayley, H. Analysis of single nucleic acid molecules with protein nanopores. In Methods in enzymology 475, 591-623 (2010).

1. A method comprising:
(a) providing (i) a nanopore system, wherein the nanopore system comprises (1) a fluid chamber and (2) a membrane comprising an artificial nanopore, wherein the membrane separates the fluid chamber into a cis side and a trans side, wherein the artificial nanopore comprises a hydrophobic portion, wherein a hydrophilic portion is coupled to the hydrophobic portion, wherein the hydrophilic portion is configured to effect translocation of at least a portion of a biopolymer through the hydrophobic portion; and
(b) translocating the at least the portion of the biopolymer through the artificial nanopore disposed within the membrane.

2. The method of claim 1, wherein the biopolymer comprises a non-nucleic acid based polymer analyte.

3. The method of claim 1, wherein the hydrophobic portion comprises a polypeptide sequence from a different protein than the hydrophilic portion.

4. The method of claim 1, wherein the artificial nanopore does not comprise at least a portion of an alpha-hemolysin protein.

5. The method of claim 1, wherein the hydrophilic portion comprises a ring forming protein, a translocation protein, a proteasome translocation domain, a pA28 monomer, a pA26 monomer, an ATPase monomer, an AAA+ translocase, a proteasome subunit, or any combination thereof.

6. The method of claim 1, wherein the hydrophobic portion comprises a transmembrane portion.

7. The method of claim 1, wherein the hydrophilic portion comprises at least one subunit of a proteasome.

8. The method of claim 1, wherein the hydrophobic portion and the hydrophilic portion are not able to form a pore as isolated polypeptides.

9. The method of claim 1, wherein the hydrophobic portion is adjacent to the hydrophilic portion.

10. The method of claim 1, wherein the hydrophobic portion is coupled to the hydrophilic portion by at least two peptide bonds.

11. The method of claim 1, wherein the hydrophobic portion is coupled to the hydrophilic portion by a linker.

12. The method of claim 1, wherein the hydrophilic portion is configured to effect speed of translocation of the at least the portion of the biopolymer.

13. The method of claim 1, wherein the hydrophobic portion comprises an α-helical forming pore or a β-barrel pore forming domain.

14. The method of claim 1, wherein the hydrophobic portion comprises a β-barrel pore forming domain.

15. The method of claim 1, further comprising detecting a current or change thereof while the at least the portion of the biopolymer is translocated through the artificial nanopore to generate a signal associated with a characteristic of the biopolymer.

16. The method of claim 15, further comprising analyzing the signal to characterize a biopolymer characteristic.

17. The method of claim 15, wherein the characteristic of the biopolymer comprises a sequence of the at least the portion of the biopolymer.

18. The method of claim 1, wherein the fluid chamber further comprises a circuitry.

19. The method of claim 18, wherein the circuitry is configured to provide a voltage between the cis side and the trans side of the fluid chamber.

20. The method of claim 1, further comprising coupling a translocase to the artificial nanopore.
